(19) United States
(12) Patent Application Publication
(10) Pub. No.: US 2023/0159873 A1
(43) Pub. Date: May 25, 2023

(54) Low-macrophage-adhesion/activation culture devices for continuous hematopoiesis and expansion of hematopoietic stem cells and progenitor cells

(71) Applicant: Schickwann Tsai, Salt Lake City, UT (US)

(72) Inventor: Schickwann Tsai, Salt Lake City, UT (US)

(21) Appl. No.: 17/971,623

(22) Filed: Oct. 23, 2022

Related U.S. Application Data
(62) Division of application No. 16/554,257, filed on Aug. 28, 2019.
(60) Provisional application No. 62/751,696, filed on Oct. 28, 2018.

(51) Int. Cl. C12M 1/00

(52) U.S. Cl. CPC C12M 23/20; C12M 23/34

(57) ABSTRACT

Hematopoietic stem cells are extremely difficult to maintain or expand in vitro. Two observations in traditional long-term bone marrow cultures strongly suggest that macrophages may be at the root of the problem: First, micromolar concentrations of hydrocortisone improve the longevity of long-term bone marrow cultures and hydrocortisone is known as a potent inhibitor of macrophage production of pro-inflammatory cytokines, chemokines, enzymes, nitrogen oxide and reactive oxygen species and redirects macrophages to the anti-inflammatory differentiation pathway; Second, the decline of hematopoiesis in long-term bone marrow cultures coincides with the development of large numbers of adherent and non-adherent macrophages including foreign body giant cells. These adherent macrophages and foreign body giant cells exhibit well-spread morphology, contain numerous lysosomes and phagolysosomes in the cytoplasm and are metabolically active. We hypothesize that hydrocortisone fails to suppress all aspects of macrophage pro-inflammatory activation/differentiation, resulting in the production of inhibitors or toxins of hematopoiesis. Macrophage adhesion in cell culture depends on serum proteins pre-adsorbed to the tissue-culture-treated polystyrene (TC-PS), which adsorbs proteins via mostly hydrophilic interactions. TC-PS is used in almost all tissue culture devices currently available. Cellular adhesion provides a strong stimulus for metabolic, mitotic and certain gene activities. Therefore, we seek to reduce macrophage adhesion and activation by culturing bone marrow cells in tissue culture devices composed of or covered with polymers with very different protein-binding characteristics than TC-PS such as polyethylene (PE) and other polyolefins, the latter bind proteins via exclusively hydrophobic interactions. As a result, polyolefins bind different proteins and in lower quantities than TC-PS. Furthermore, PE does not contain additional chemical features like the phenolic rings of polystyrene that might contribute to protein binding and macrophage adhesion/activation. Using these new culture devices, we developed a drastically different long-term bone marrow culture, the “Low Macrophage-Adhesion/Activation” (LoMAC) bone marrow culture. In LoMAC bone marrow culture, hematopoiesis continues for months to over a year and hematopoietic stem cells are amplified gradually. In stark contrast to traditional long-term bone marrow cultures, de novo erythropoiesis and megakaryocytopoiesis proceed robustly in the LoMAC bone marrow culture and B-lymphocyte and natural killer cell progenitors can be continuously derived. Thus, these new culture devices and the associated LoMAC culture method offer a new way to study hematopoiesis in vitro and provide a more robust platform for the expansion of hematopoietic stem cells and progenitors ex vivo.

### CROSS REFERENCE TO RELATED APPLICATIONS:

The present application is a division of the inventor's U.S. application Ser. No. 16/554,257 filed Aug. 29, 2019, entitled “Low-macrophage-adhesion/activation culture devices and methods thereof for continuous hematopoiesis and expansion of hematopoietic stem cells”, currently pending, which claims the benefit of U.S. Provisional Application Ser. No. 62/751,696 filed Oct. 28, 2018, the content of which is relied upon and incorporated herein by reference.

### FEDERALLY SPONSORED RESEARCH

None.

### SEQUENCE LISTING

None.

### TECHNICAL FIELD

This invention relates to new tissue culture devices in which all internal surfaces in temporary or permanent contact with the cultured cells are covered with or composed of materials designed to reduce the adhesion and pro-inflammatory activation of macrophages and the subsequent production of cytokines, chemokines, lytic enzymes, nitric oxide (NO), reactive oxygen species (ROS) and other phagocytes-produced factors that are potentially harmful to hematopoietic stem and progenitor cells. The new culture devices provide the physical foundation for creating a non-inflammatory or anti-inflammatory culture environment in which hematopoiesis can continue for several months with expansion of hematopoietic stem cells and de novo production of most types of blood cells and progenitors including red blood cells and megakaryocytes. Furthermore, the starting population can be unpurified (e.g. whole bone marrow) or partially purified or purified populations of bone marrow (BM), cord blood (CB) or peripheral blood stem/progenitor cells (PBSC). Lengthy purification of hematopoietic stem cells or selective purging of certain white blood cells is not necessary. The Low-Macrophage Adhesion/Activation (“LoMAC”) culture devices and the associated culture methods work together to support the survival and proliferation of hematopoietic stem cells and progenitor cells ex vivo over a long period of time.

### BACKGROUND OF THE INVENTIOIN

Hematopoietic stem cells (HSC) provide lifetime production of all types of blood cells by virtue of their capacity for self-renewal and for differentiation into lineage-restricted progenitors that can proliferate extensively before terminal differentiation. While it has become relatively easy to culture most types of hematopoietic progenitors in short-term (1-2 weeks) assays, it remains very challenging to maintain, let alone expand, HSC in vitro for more than 2-3 weeks.

The best-known method for long-term cultivation of BM is the “long-term bone marrow culture” (LTBMC) or Dexter culture method (1). The current protocol for LTBMC involves two sequential steps. In the first step, an adherent stromal layer consisting of macrophages, fibroblastoid stromal cells (also referred to as mesenchymal cells), endothelial cells, osteoclasts and osteoblasts is established by culturing whole marrow in “tissue-culture-treated” polystyrene (TC-PS) dishes, cluster plates or tissue culture flasks for 2-4 weeks. The established stromal layer is then irradiated to kill all lingering hematopoietic cells and recharged with a second inoculum of bone marrow that provides the starting HSC and progenitors for LTBMC. To improve the longevity of LTBMC, both the establishment of the stromal layer and the maintenance of LTBMC are performed at 33° C. instead of 37° C. Initially, only pre-screened batches of horse sera (HS) were found to be capable of supporting LTBMC. Later, it was discovered that the addition of hydrocortisone (HC) at 10⁻⁵ to 10⁻⁶ M during the establishment of the stromal layer and the maintenance of LTBMC allowed the use of most batches of HS and fetal bovine serum (FBS), although the exact roles of HC in LTBMC were not fully understood (2). However, available evidence suggests that the beneficial effects of HC in LTBMC are derived mainly from its anti-inflammatory activity on phagocytes (macrophages, monocytes and neutrophils)(infra).

The monocyte-macrophage lineage is remarkable for its diversity and plasticity in functional phenotypes, with the M1 (also know as “pro-inflammatory” or “classically activated”) and M2 (also know as “anti-inflammatory” or “alternatively activated”) differentiation/activation states representing the polar extremes of a wide spectrum of differentiation states (3-5). Many studies on the activation or differentiation of macrophages (and monocytes and neutrophils) during microbial infection and foreign-body response have shown that HC can reprogram macrophages (or monocytes or neutrophils) from M1 pro-inflammatory activation/differentiation state characterized by the production of pro-inflammatory mediators to an M2 anti-inflammatory state characterized by the production of anti-inflammatory mediators such as interleukin-10 (IL-10) and tissue inhibitor of metalloprotease-1 (TIMP-1) (3-6). Since many inflammatory cytokines, chemokines, NO and ROS secreted by M1 macrophages (or monocytes or neutrophils) have inhibitory, toxic or pro-apoptotic effects on hematopoietic stem cells and progenitors (7-10), the M1-to-M2 activation/differentiation switch of macrophages (or monocytes or neutrophils) can account for many of the beneficial effects of HC in LTBMC. The hypothermic temperature (33° C.) further reduces cellular metabolism and mitotic activities in general and the activities of macrophages in particular and thereby improves the longevity of LTBMC.

Although LTBMC has been reported to support long-term hematopoiesis for several months at 33° C. in very experienced hands, the more common experience is that it is an attrition or run-down system in which HSC decline rapidly over 2-6 weeks after the initial wave of production of monocytes, macrophages and neutrophils. This decline is accelerated when the culture is maintained at 37° C. Available data indicate that the LTBMC system cannot support HSC renewal (11). The addition of hematopoietic growth factors such as stem cell factor (SCF or c-kit ligand or KL), granulocyte-macrophage colony stimulating factor (GM-CSF) and interleukin-3 (IL-3) does not improve the longevity of LTBMC. Furthermore, hematopoiesis in LTBMC is skewed overwhelmingly towards myelopoiesis (production of monocytes, macrophages and neutrophils)(1). De novo erythropoiesis beyond the stage of burst-forming unit-erythroid (BFU-E; the most primitive erythroid progenitor detectable by colony assay in semi-solid medium) or megakaryocytopoiesis beyond the stage of colony-forming unit-megakaryocyte (CFU-Meg) is rarely seen after the first 2-5 weeks even in the presence of high concentrations of erythropoietin (EPO) or thrombopoietin (TPO). Furthermore, the frequencies of BFU-E and CFU-Meg are very low or undetectable in weeks-old LTBMC (12). Taken together, these observations suggest that the environment of LTBMC is not conducive to the survival and/or proliferation of HSC or erythroid or megakaryocytic progenitors and raise the possibility that inhibitors or cytotoxins of hematopoiesis are generated in traditional LTBMC.

In the course of investigating the roles of stromal cells in LTBMC, we noticed that the rapid decline of hematopoiesis in LTBMC coincided temporally with the development of large numbers of macrophages, monocytes and neutrophils during the first 2-4 weeks. While mouse neutrophils die 4-7 days after their maturation, macrophages can survive for weeks to months. Some macrophages retain the capacity for limited mitosis. Thus, the number and impact of macrophages exceeds those of neutrophils in traditional LTBMC. Most of the macrophages in established LTBMC adhere firmly to the tissue culture surface or to the extracellular matrix of stromal cells. These adherent macrophages display well-spread or elongated (migrating) morphology with “foamy” cytoplasm due to the presence of numerous lysosomes, phagosomes, phagolysosomes, inclusion bodies (of phagocytosed cellular remnants), endocytic and secretory vesicles in the cytoplasm. Many adherent and non-adherent macrophages fuse together to form multinucleated “foreign body giant cells” (FBGC; also known as “multinucleated giant cells” or MNG). In LTBMC, the adhering of macrophages to tissue culture surface and their subsequent development into FBGC take place despite the presence of high concentrations (10⁻⁵ to 10⁻⁶ M) of HC. In fact, adherent macrophages including FBGC are the most prominent and consistent feature in LTBMC. These macrophages display many features of classically activated macrophages such as cytoplasmic lysosomes, phagosomes, phagolysosomes and secretion of pro-inflammatory cytokines and chemokines, albeit at lower levels than classical M1 macrophages that have been primed with interferon-y and then stimulated with lipopolysaccharide. In addition to adherent macrophages and FBGC, there are many nonadherent monocytes and macrophages that undoubtedly contribute to the overall effect. Fibroblastoid stromal cells usually start out in small numbers and gradually increase in number over several weeks. Their development lags behind that of monocytes and macrophages by 2-3 weeks. Fibroblastoid stromal cells eventually become senescent after several weeks to months. The effects of monocytes and macrophages are amplified in a positive feed-back loop by fibroblastoid stromal cells as the latter can secret large amounts of macrophage-colony stimulating factor (M-CSF) and other hematopoietic growth factors (e.g. GM-CSF, G-CSF) and cytokines (e.g. interleukin 4 or IL-4, IL-6, IL-9, IL-11) in response to pro-inflammatory cytokines such as tumor necrosis factor-α (TNF_{α}) and interleukin-1_{β} (IL-1_{β}) released by monocytes and macrophages (13, 14).

Given the temporal correlation between the development of large numbers of adherent macrophages and FBGC and the decline in hematopoiesis in LTBMC, we considered the possibility that HC fails to convert all M1 macrophages to M2 macrophages or completely abolish the pro-inflammatory activities of M1 macrophages in traditional LTBMC established in TC-PS-based tissue culture devices. This is likely as monocytes/macrophages in LTBMC represent a mixture of preexisting BM monocytes/macrophages and de novo generated monocytes/macrophages with different developmental histories and functional states. Furthermore, virtually all studies on macrophage activation/differentiation have been conducted in tissue culture devices made of tissue-culture-treated polystyrene (TC-PS) and therefore the roles of TC-PS in macrophage activation/differentiation in vitro may have been overlooked.

The majority of classically activated M1 macrophages adhere to the TC-PS tissue culture surface, display well-spread or elongated morphology with foamy cytoplasm and are phagocytic. Adhesion, e.g. via integrin receptors, is a potent stimulus for most cells including macrophages and can trigger profound changes in metabolic activities, gene expression and differentiation pathways. After unsuccessful attempts to ingest the plastic (the “foreign body”), adherent M1 macrophages may mount a “frustrated phagocyte response” in which macrophages (or other phagocytes) secret a host of hydrolytic enzymes, acids, NO and ROS into the space between the macrophages (or phagocytes) and the PS or TC-PS tissue culture surface (15). Macrophages may fuse together to form large, multinucleated FBGC in order to ingest large foreign bodies (>10 μm). FBGC are numerous three to four weeks after the start of LTBMC in the presence of 10⁻⁵-10⁻⁶ M HC. The substances secreted by frustrated macrophages and FBGC are essentially the same ones that macrophages normally secret into lysosomes and phagolysosomes during microbial infections in an effort to kill the invading microorganisms. Some of the secreted substances leak into the culture medium and may harm or kill HSC and progenitors. One key cytokine secreted by M1 activated macrophages is tumor necrosis factor-α (TNF_{α}), which binds to TNF receptor (TNFR) and triggers apoptosis of many types of hematopoietic progenitors. Numerous studies have shown that TNF_{α} has potent inhibitory or toxic effects on hematopoietic progenitors (7,8). Thus, TNF_{α} may have particular relevance in the demise of hematopoietic stem and progenitor cells in LTBMC.

The sequence of events outlined above provides a potential explanation for the decline of hematopoietic stem and progenitor cells in LTBMC following the development of large numbers of adherent and non-adherent macrophages. The adherence of macrophage and other cells (e.g. monocytes, neutrophils and fibroblasts) to the tissue culture surface depends on serum proteins such as fibrinogen, fibronectin, vitronectin, immunoglobulins, complements and albumin that have been adsorbed to the tissue culture surface (typically TC-PS) via a combination of hydrophobic, hydrophilic and ionic interactions. Different hydrocarbon polymers exhibit different affinities for different serum proteins and the differences in adsorbed proteins can influence the adhesion and/or activation and/or differentiation of macrophages (16, 17). Most tissue culture devices are made of PS that has been treated with oxygen plasma or other materials (e.g. peptides, proteins and extracellular matrix) to render it more negatively charged and more hydrophilic. Indeed, it has been shown that monocytes cultured on the hydrophilic TC-PS surface undergo predominantly M1 activation/differentiation with secretion of pro-inflammatory cytokines and active phagocytosis while those cultured on the untreated, more hydrophobic PS surface undergo mostly M2 differentiation with secretion of anti-inflammatory cytokines and no phagocytosis (17). Last but not least, we cannot rule out a priori the possibility that certain chemical features such as the repeating phenolic rings of PS or TC-PS may contribute to macrophage activation/differentiation via pattern-recognition receptors independent of its role in protein binding. Virtually all studies of M1 vs. M2 activation/differentiation treat the PS or TC-PS culture surface as inert material despite the fact that it represents a very large “foreign body” to macrophages.

One way to test our hypotheses on the possible effects of tissue culture surface on macrophage adhesion/activation and the subsequent impact on hematopoiesis in LTBMC is to perform bone marrow culture in tissue culture devices with a culture surface that differs significantly from TC-PS in terms of hydrophilicity, protein binding, electrical charges and cell adhesion. In this regard, the hydrocarbon polymer polyethylene (PE; also known as polyethene)(18) consists of linear chains of carbon atoms linked to hydrogen atoms ([—CH₂—CH₂—]ₙ) and is devoid of any additional chemical features such as the phenolic rings of PS (19). The interaction of PE with proteins is through hydrophobic interactions exclusively while interaction with TC-PS is mainly through hydrophilic interactions. Therefore, they are expected to have very different protein adsorption profiles. In general, PE has low affinities for proteins compared to TC-PS. Some estimates put the protein binding capacity of PE at one half to one tenth that of PS overall, although it clearly depends on the protein species. PE is in fact the simplest hydrocarbon polymer possible and a candidate material for producing alternative tissue culture devices with a low potential for protein binding and macrophage adhesion/activation. However, PE has been considered unsuitable for making tissue culture dishes or cluster plates or flasks because of thickness-dependent opacity, pliability, low protein/cell binding and troublesome molding characteristics. PS, on the other hand, is used in almost all current tissue culture dishes or cluster plates or flasks due to its transparency, rigidity, excellent molding characteristics, high protein-binding capacity and convenient sterilization by irradiation. PS-based tissue culture devices are usually “tissue-culture-treated” (e.g. by corona discharge under atmospheric conditions or O₂ plasma under vacuum to incorporate more O₂ into PS so that the surface becomes hydrophilic and negatively charged) or by coating with polypeptides (e.g. poly-lysine or arginine-glycine-aspartic acid/RGD) or proteins (e.g. collagen, fibronectin, vitronectin) or extracellular matrix components to further enhance cell adhesion. In contrast, PE has never been used in the production of tissue culture dishes, cluster plates or flasks due to its low protein- and cell-binding capacities, thickness-dependent opacity and troublesome molding.

In this application, we introduce a drastically different long-term BM culture using newly developed PE-coated tissue culture devices or devices fabricated completely from PE-like materials (e.g. other polyolefins or their copolymers) designed to minimize macrophage adhesion/activation while preserving some, albeit low, levels of adhesion for HSC and progenitors. To avoid accidental or transient activation of macrophages during the cultivation and manipulation of the BM cells, the entire bottom AND sidewall surfaces of the tissue culture devices are covered with a thin (100-200 micron) membrane of PE. Alternatively, the entire device is fabricated from PE-like material (in terms of atomic composition, hydrophobicity, protein binding) but with high transparency and shape retention such as poly(4-methyl-1-pentene) (PMP). As demonstrated below in Embodiments, macrophages adhered poorly to PE (or other polyolefins) culture surface and did not undergo pro-inflammatory activation effectively as they would on TC-PS culture surface. They are also non-phagocytic. In most comparisons, hydrocortisone (HC) was included to further suppress macrophage pro-inflammatory activation. In this unique “Low-Macrophage-Adhesion/Activation” (abbreviated as LoMAC) culture environment, HSC can be maintained and expanded for many months in complete absence of a stromal layer. Importantly, hematopoiesis declined quickly when established BM LoMAC cultures were transferred to TC-PS culture devices, thus proving that TC-PS is a critical factor in the decline of hematopoiesis in LTBMC. Interestingly, de novo erythropoiesis and megakaryocytosis proceed robustly and continuously (with EPO and TPO) in the liquid phase of mouse LoMAC culture. This degree of de novo erythropoiesis and megakaryocytopoiesis has never been observed in traditional LTBMC using TC-PS culture devices. In fact, the progenitors (HSC, BFU-E, CFU-Meg) and precursors (erythroblasts and megakaryocytes) that predominate in mouse LoMAC bone marrow cultures set up in PE-coated culture devices are exactly those missing from traditional mouse LTBMC. Further observations using other tissue culture devices with ultra-low cell binding indicates that across-the-board inhibition of cellular adhesion is not sufficient for creating a permissible environment for long-term hematopoiesis in vitro. The chemistry of the culture surface also matters.

### BRIEF SUMMARY OF THE INVENTION

This invention relates to new tissue culture devices (e.g. dishes, cluster plates, flasks, tubes, bags and bioreactors) in which both the bottom and sidewalls are covered with low-density polyethylene (LDPE) or linear low-density polyethylene (LLDPE) with the objective of reducing the adherence and pro-inflammatory activation/differentiation of macrophages (as well as monocytes and neutrophils) and their subsequent production of pro-inflammatory mediators that are harmful to hematopoietic stem and progenitor cells. Alternatively, the entire device is fabricated from polyolefins that exhibit PE-like properties such as hydrophobicity and low protein/cell binding but with high degrees of transparency and shape retention. An associated method for continuous hematopoiesis and expansion of hematopoietic stem and progenitor cells over several months in the “Low Macrophage-Adhesion/Activation” culture environment is described.

### DRAWINGS

FIG. 1 is a removable single-well LDPE insert with a holed roof at the top.

FIG. 2 is a removable single-well LDPE insert with a retainer flange at the top.

FIG. 3 is a removable 6-well LDPE insert with 6 deep wells and a flat top connecting the wells.

FIG. 4 shows three different-sized culture dishes with a “super-deep dish” design in which both the bottom and sidewall culture surfaces are covered with LDPE or the entire dish is fabricated from transparent, rigid polyolefin such as PMP.

FIG. 5 compares the adhesion of the OP-9 fibroblast stromal cell line on PS vs. TC-PS (tissue culture-treated PS) vs. PE surfaces.

FIG. 6 compares the adhesion of the macrophage-like WEHI 3B cell line on PS vs. TC-PS vs. PE surfaces.

FIG. 7 compares the growth curves of the macrophage-like WEHI 3B in TC-PS vs. PE-coated tissue culture devices over 21 days.

FIG. 8 compares the growth curves of mouse bone marrow cultured in TC-PS vs. PE-coated tissue culture devices over the first 48 days.

FIG. 9A is a phase-contrast micrograph of a day 18 mouse BM culture established in a traditional TC-PS tissue culture dish.

FIG. 9B. is a phase-contrast micrograph of a day 18 mouse BM culture established in a PE-coated tissue culture dish.

FIG. 10 compares the percentages of macrophages with inclusion bodies (result of phagocytosis) in 12-day-old mouse bone marrow cultures established in TC-PS vs. PE-coated wells. FIG. 11 compares the level of TNF_{α} in mouse bone marrow cultures established in TC-PS vs. PE-coated wells without or with hydrocortisone.

FIG. 12 compares the numbers of CAFC_{d35} of mouse bone marrow cultured in TC-PS vs. PE-coated tissue culture devices over 0, 12, 24 and 36 days.

FIG. 13 compares the numbers of CAFC_{d35} of mouse bone marrow cultured in devices with TC-PS vs. PE-coated tissue culture devices using more starting cells and less frequent sampling than the cultures in FIG. 12.

FIG. 14 compares the numbers of CAFC_{d35} of day 5 post-5-fluorouracil mouse bone marrow cultured in TC-PS vs. PE-coated tissue culture devices.

FIG. 15A shows a Wright-Giemsa stained cytospin preparation of a 160-day-old LoMAC (low-macrophage adhesion/activation) culture of mouse bone marrow before exposure to erythropoietin.

FIG. 15B shows Wright-Giemsa stained cytospin preparations of cells in a 160-day-old LoMAC (low-macrophage adhesion/activation) culture of mouse bone marrow after exposure to erythropoietin for 16 days.

FIG. 16 compares the growth curves of a 260-day-old LoMAC (low-macrophage adhesion/activation) culture of mouse bone marrow that was subsequently transferred to TC-PS vs. PE-coated culture devices.

FIG. 17A is a phase-contrast micrograph of an 80-day-old LoMAC culture of human bone marrow showing an area with macrophages alone.

FIG. 17B is a phase-contrast micrograph of an 80-day-old LoMAC culture of human bone marrow showing an area with macrophages plus active hematopoiesis.

FIG. 17C is a phase-contrast micrograph of a 100-day-old, spent LoMAC culture of human bone marrow showing (mostly non-adherent) macrophages and numerous apoptotic bodies.

### BRIEF DESCRIPTION OF DRAWINGS

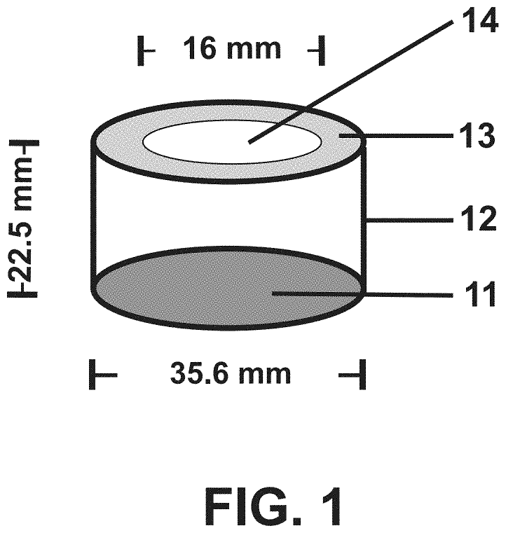

FIG. 1 is a removable single-well (for 6-well plates) LDPE insert (100-200 μm thick) with a holed roof at the top. The holed roof helps maintain the overall shape of the insert and reduces evaporation of culture medium. The roof is laminated with other plastic materials such as PS, PE or polypropylene (PP) to increase its rigidity and durability.

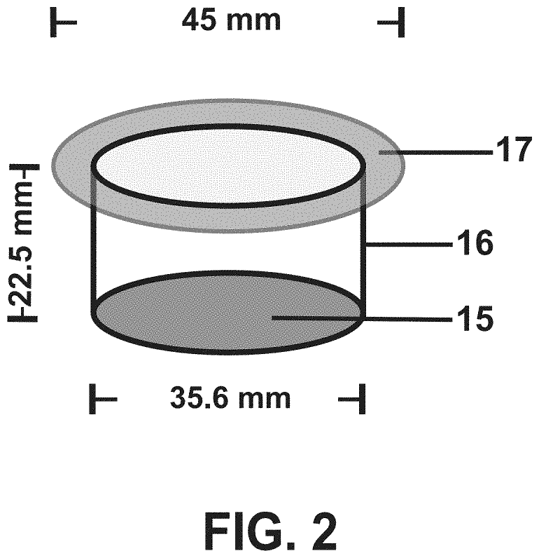

FIG. 2 is a removable single-well (for 6-well plates) LDPE insert (100-200 ρm thick) with a retainer flange at the top. The retainer flange is laminated with other plastic materials such as PS, PE or PP to increase its rigidity and durability.

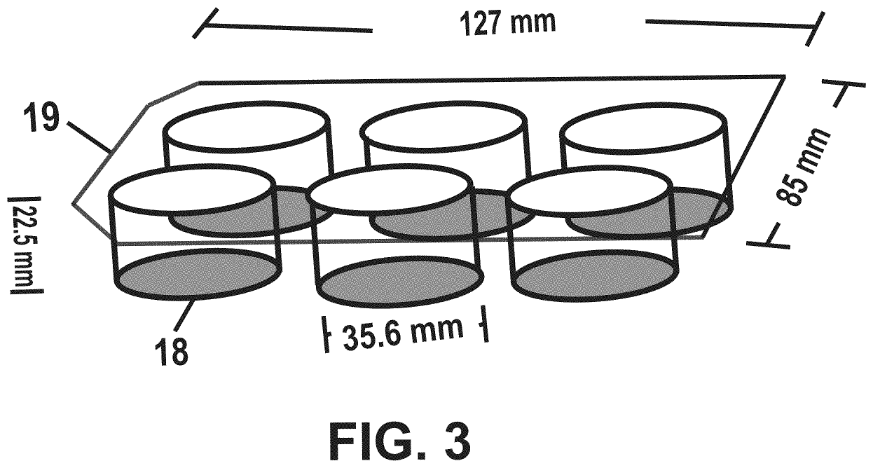

FIG. 3 is a removable 6-well LDPE insert (100-200 μm thick) with 6 deep wells. Each well is about 22.5 mm deep in order to accommodate more culture medium and minimize disturbance or loss of cells at the bottom during frequent media exchange. The flat connecting top between wells is laminated with other plastic materials such as PS, PE or PP to increase its rigidity and durability. The 6-well insert fits inside a matching 6-well plate (PS or TC-PS) with deep wells and can remain removable or affixed to the 6-well plate with adhesives.

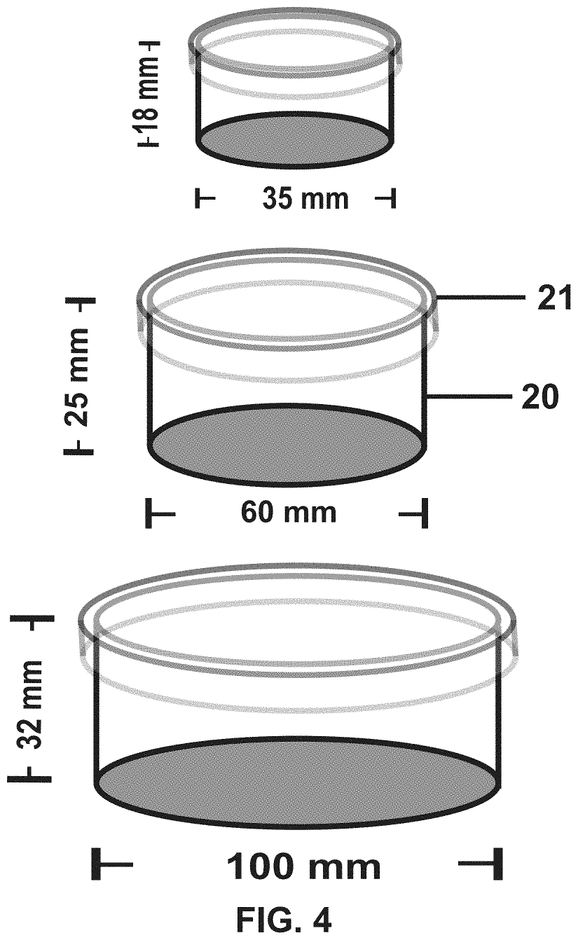

FIG. 4 shows three different-sized (35, 60 and 100 mm in diameter) PS culture dishes with a “super-deep dish” design in which both the bottom and sidewall culture surfaces are covered with LDPE or LLDPE (100-200 μm thick). The “super-deep dish” design must have a dish height-to-bottom diameter ratio greater than 0.2 to allow the use of more culture medium in order to provide more nutrients and buffering capacity, avoid contamination and reduce disturbance and loss of hematopoietic cells at the bottom during media exchange. Actual ratios for the 35-, 60- and 100-mm dishes shown are 0.51, 0.42 and 0.32, respectively. Alternatively, the entire super-deep dish can be injection-molded from hydrocarbon polymers with protein binding and cell adhesion properties similar to those of LDPE but exhibiting better transparency and shape retention such as poly(4-methyl-1-pentene)(PMP). All measurements shown are external dimensions.

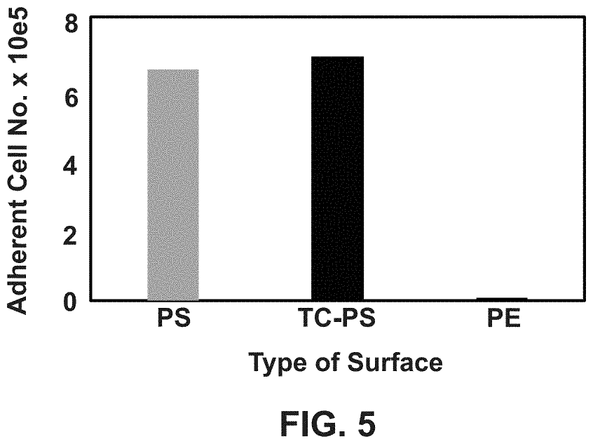

FIG. 5 is a bar graph comparing the adhesion of the OP-9 fibroblast stromal cell line on PS vs. TC-PS vs. PE surfaces. A total of 4×10⁵ OP-9 cells were suspended in 4 ml of DME supplemented with 10% (vol./vol.) FBS and 5×10⁻⁵ M 2-ME and added to each well of a 6-well plate with a PS, TC-PS or LDPE culture surface and incubated at 37° C. for 24 hr. Cultures were then washed with PBS three times. Adherent cells were detached by treatment with trypsin-EDTA at 37° C. for 5 min. and counted. OP-9 cells adhered with 100% efficiency to both PS and TC-PS surfaces but extremely poorly (0.11×10⁵ or 2.8% of input) to the LDPE surface. Please note that one to two cell divisions had occurred during the incubation period, resulting in increased cell numbers. Data represent the means of duplicates.

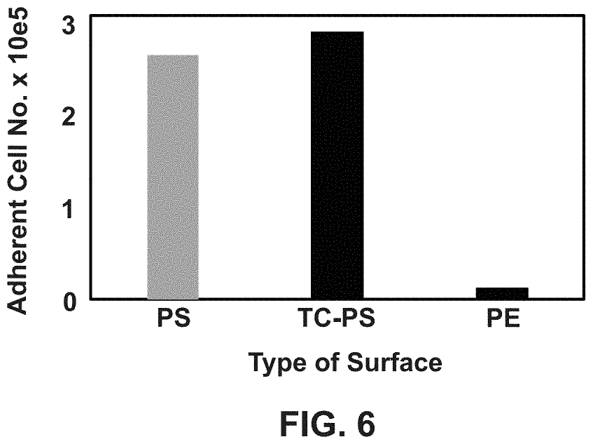

FIG. 6 is a bar graph comparing the adhesion of the WEHI 3B macrophage-like cell line on PS vs. TC-PS vs. PE surfaces. 3.0×10⁵ WEHI 3B cells were suspended in 4 ml of DME supplemented with 10% (vol./vol.) FBS and 5×10⁻⁵ M 2-ME and added to each well of a 6-well plate with a PS, TC-PS or LDPE culture surface and incubated at 37° C. for 24 hr. Cultures were washed with PBS three times to remove non-adherent cells. Adherent cells were detached by treatment with trypsin-EDTA at 37° C. for 20 min. and flushed vigorously and counted. WEHI 3B adhered rapidly to both PS and TC-PS surfaces but very poorly (0.02×10⁵ or 0.7% of input) to LDPE surface. Data are means of duplicates.

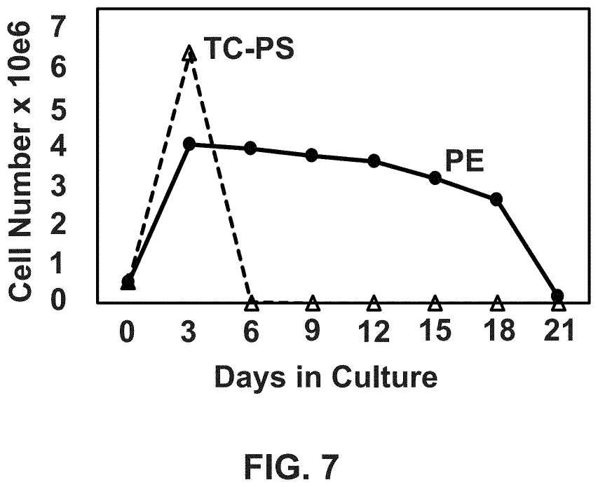

FIG. 7 is a comparison of the growth curves of the WEHI 3B macrophage-like cell line in devices with TC-PS vs. PE culture surfaces. 0.5×10⁶ WEHI 3B cells were suspended in 5 ml of DME supplemented with 10% (vol./vol.) FBS and 5×10⁻⁵ M 2-ME and cultured in each well of a 6-well cluster plate with a TC-PS vs. PE culture surfaces. Total cell numbers (adherent plus non-adherent) were counted on days 3-21. No fresh medium was added or exchanged for the entire culture period (21 days). WEHI 3B cultured in TC-PS wells (broken line) grew rapidly to the peak density in three days and then died rapidly due to exhaustion of nutrients and acidity (pH<6.7) with all cells dead by day 6. In contrast, WEHI 3B grown in LDPE-coated wells (solid line) were able to slow down metabolism and mitoses and became quiescent as the nutrients became depleted. As a result, the pH of the medium in the PE group remained above >7.0 for 21 days even though no fresh medium was added or exchanged.

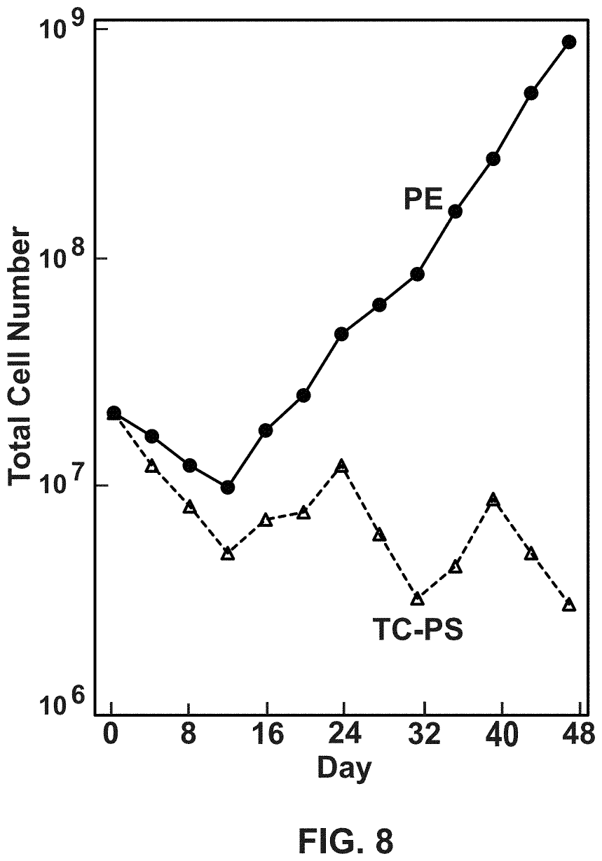

FIG. 8 compares the growth curves of BM cultured in TC-PS (negative control; broken line) vs. PE-coated (solid line) wells. Each culture was started with 2.2×10⁷ (TNC) BM cells of a 20-week-old male C57BL/6 mouse supplemented with 4% BHK/KL conditioned medium (equivalent of 20-40 ng/ml mKL), 4% BHK/TPO conditioned medium (equivalent of 20-40 ng/ml mTPO) and 10⁻⁶ M HC. One third of culture medium was exchanged every 2 days. The cultures were subdivided at a 1:2 ratio when cell numbers exceeded 1.0-1.2×10⁷ per well after week 2. Cells in PE-coated wells continued to expand in a quasi-logarithmic manner beyond day 100. In contrast, the control cultures in TC-PS wells were spent after day 50. The vertical axis is on a logarithmic scale. Data are means of duplicates.

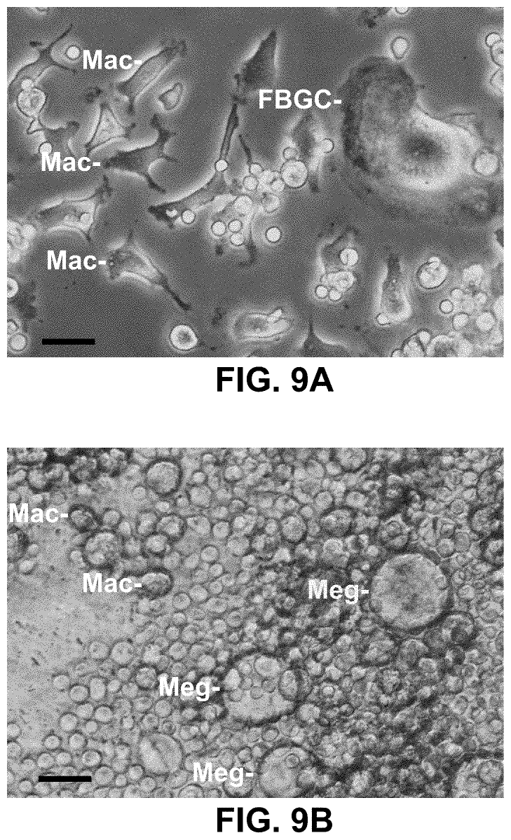

FIG. 9 shows phase-contrast micrographs of mouse BM cultures established in TC-PS vs. PE-coated tissue culture devices. FIG. 9A is a phase-contrast micrograph of the control culture established in a TC-PS well on day 18 of the culture. Three adherent macrophages with well-spread cytoplasm are labeled “Mac”. The “FBGC” label identifies a foreign body giant cell. Small-to-medium round refractile cells are monocytes, neutrophils and progenitors. FIG. 9B is a phase-contrast micrograph of a day 18 LoMAC culture established in a LDPE-coated well. Megakaryocytes are labeled “Meg” and macrophages are labeled “Mac”. Remaining small-to-medium round refractile cells are monocytes, neutrophils and progenitors at all stages of differentiation. Please note that adherent macrophages with well-spread cytoplasm or FBGC are extremely rare in LoMAC cultures and none is visible in this field. Bars=50 μm.

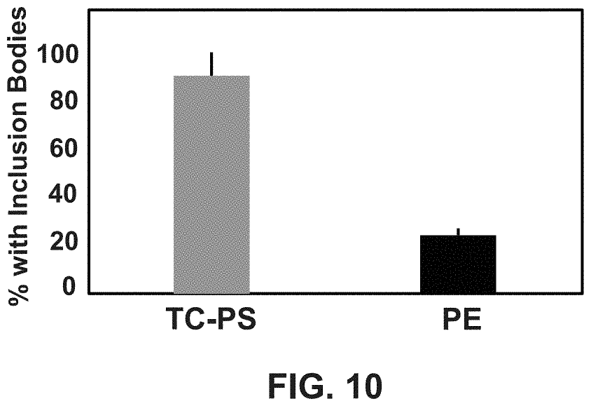

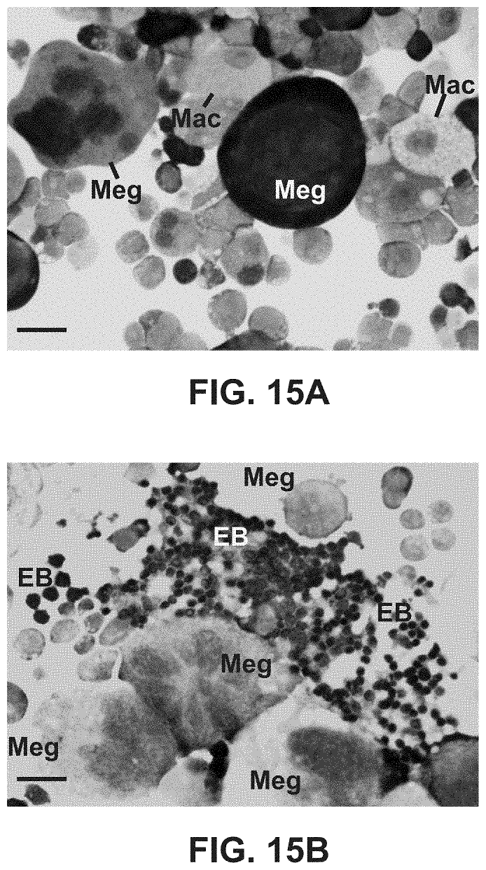

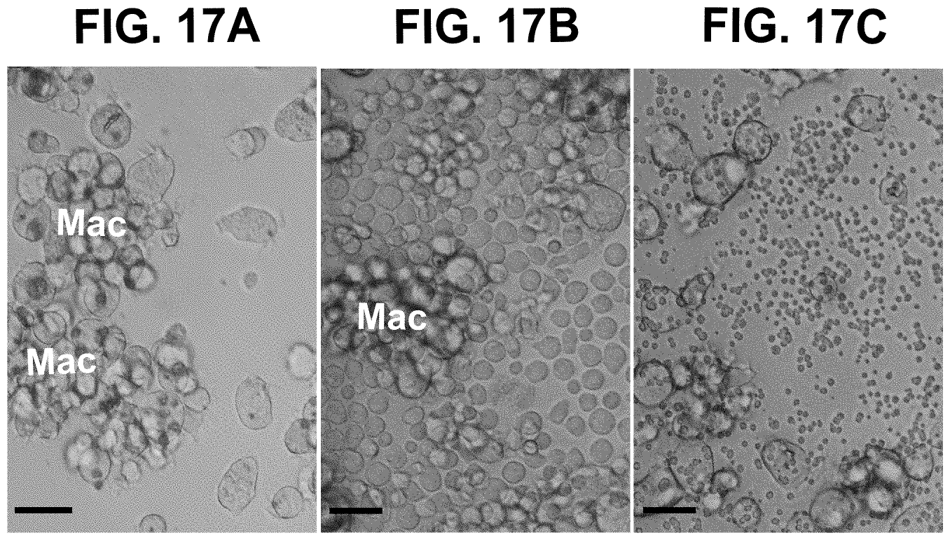

FIG. 10 is a comparison of the percentages of macrophages with inclusion bodies (as a measure of phagocytic activity) in 12-day-old mouse bone marrow cultures established in TC-PS vs. PE-coated wells. PE decreased the phagocytic activities of pre-existing bone marrow macrophages significantly and nearly completely of de novo generated macrophages in LoMAC cultures (see FIG. 15A and FIG. 17C below). Data are means of triplicates.

FIG. 11 is a bar graph comparing the levels of TNF_{α} (determined by ELISA) in 3-day conditioned media of 12-day-old mouse bone marrow cultures established in TC-PS vs. PE-coated devices without or with HC. Both LDPE and HC decreased the production of TNF_{α}. Data are means of triplicates.

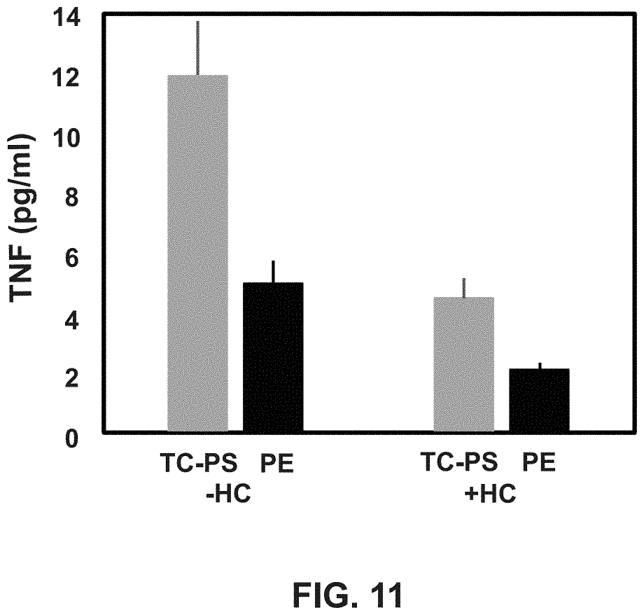

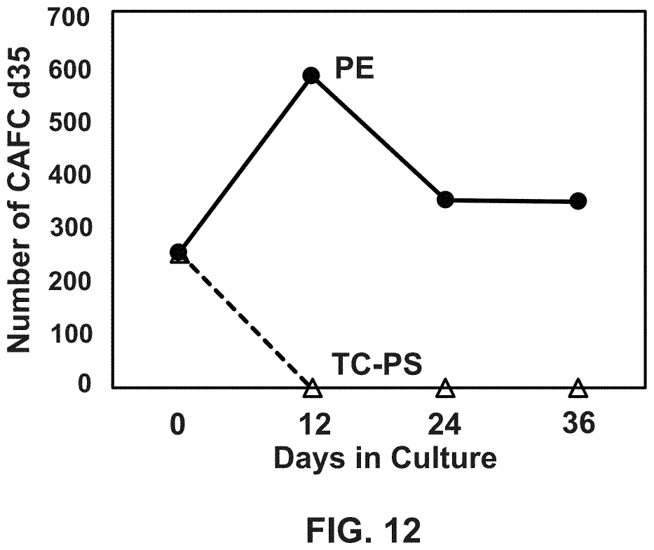

FIG. 12 is a comparison of the numbers of CAFC_{d35} of mouse bone marrow cultured in a 6-well plate with TC-PS vs. PE-coated culture surface on day 0, 12, 24 and 36. Each culture was started with 1.0×10⁷ (TNC) BM cells from a 6-week-old male C57BL/6 mouse and 6-ml of IMDM/RPMI (1:1 mix) medium supplemented with 20% (vol./vol.) HS, 5×10⁻⁵ M 2-ME, 1×10⁻⁶ M HC, penicillin/streptomycin, BHK/KL conditioned medium (4% vol./vol.) as a source of mKL and BHK/TPO conditioned medium (4% vol./vol.) as a source of mTPO. CAFC assays were performed on day 0, 12, 24 and 36 in 12-well plates with preformed OP-9 stromal layers that had been treated with mitomycin C (8 μg/ml) for two hr. and washed twice with PBS. Cultured marrow cells were harvested and washed to remove HC and hematopoietic growth factors before CAFC assay. Four different cell doses were plated with a minimum of 6 wells per cell dose. The culture medium consisted of IMDM/RPMI (1:1 mix) medium supplemented with 15% (vol./vol.) HS, 5×10⁻⁵ M 2-ME and penicillin/streptomycin. One third of the medium (2 ml/well) was replaced every 3 days or whenever the medium became acidic. Cobblestone areas were counted on day 35 using an inverted microscope with phase contrast and a 20× objective and a 10× ocular. Numbers of CAFC_{d35} were calculated using the maximum likelihood method. Virtually all CAFC_{d35} in bone marrow cultured in standard TC-PS wells (broken line) disappeared by day 12 with no CAFC_{d35} detected on day 24 or 36. In contrast, BM cultured in PE-coated wells (“LoMAC” culture; solid line) increased on day 12 and remain elevated on day 24 and 36. They remained detectable for many months in bone marrow cultures performed in LDPE-coated wells (not shown).

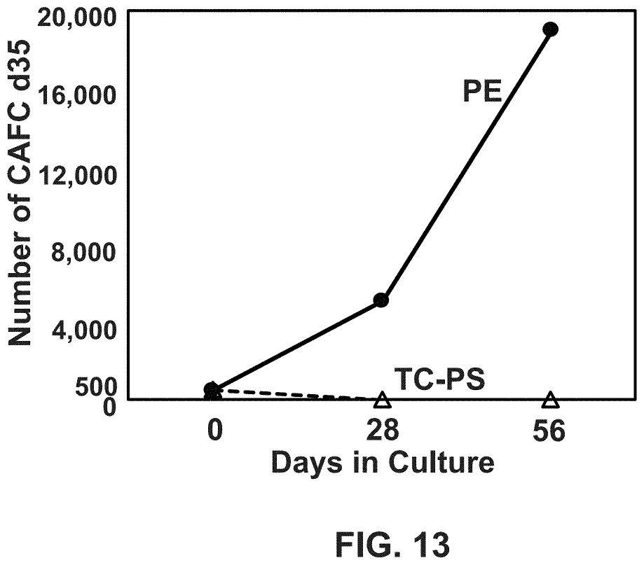

FIG. 13 is a comparison of CAFC_{d35} numbers on day 0, 24, and 56 of mouse bone marrow cultured in TC-PS vs. PE-coated culture devices. These cultures were established with more BM cells (2.2×10⁷ TNC per well) from a 20-week-old male C57BL/6 mouse than the experiment shown in FIG. 12. The culture was also subject to less frequent manipulation or disturbance than the cultures shown in FIG. 12. As a result, HSC expansion was more pronounced in the culture established in LDPE-coated culture device (“LoMAC” culture; solid line). The control cultures established in TC-PS culture device showed rapid decline of HSC with none detected on days 28 and 56 (broken line).

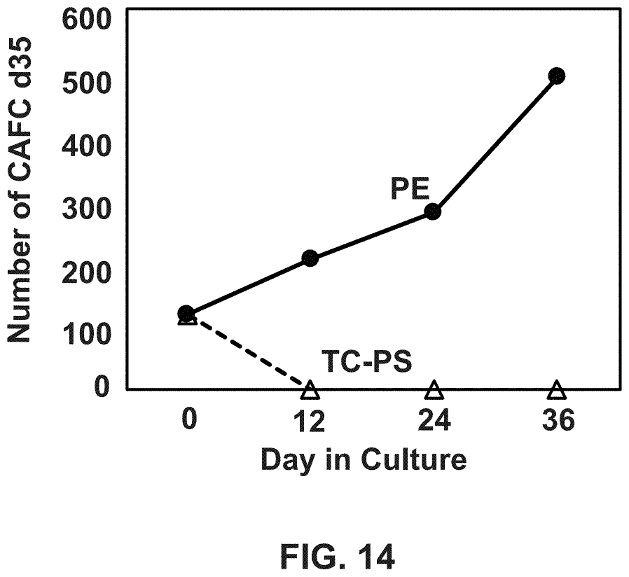

FIG. 14 is a comparison of the numbers of CAFC_{d35} of post-5 fluorouracil mouse bone marrow cultured in a 6-well plate with TC-PS vs. PE-coated culture surface on day 0, 12, 24 and 36. A 6-week-old male C57BL/6 mouse was injected intraperitoneally with 5-fuorouracil (SoloPak Laboratories) at 150 mg/Kg body weight 5 days before bone marrow harvest. 5-fluorouracil kills all rapidly cycling BM progenitors but spares quiescent or non-dividing HSC. Each culture was started with one-bone (each femur or tibia was treated as one bone) equivalent of post-5-fluorouracil bone marrow and 6-ml of IMDM/RPMI (1:1 mix) medium supplemented with 20% (vol./vol.) HS, 5×10⁻⁵ M 2-ME, 1×10⁻⁶ M HC, penicillin/streptomycin, BHK/KL conditioned medium (4% vol./vol.) as a source of mKL and BHK/TPO conditioned medium (4% vol./vol.) as a source of mTPO. CAFC assays were performed on day 0, 12, 24 and 36 in 12-well plates with preformed OP-9 stromal layers that had been treated with mitomycin C as described in the legend of FIG. 12. Virtually all CAFC_{d35} in bone marrow cultured in standard TC-PS wells (broken line) disappeared by day 12 with no CAFC_{d35} detected on day 24 or 36. In contrast, post-5-fluorouracil BM cultured in LDPE-coated wells (the “LoMAC” culture; solid line) showed continuous expansion of CAFC_{d35} on days 12, 24 and 36 and beyond.

FIG. 15 shows the morphology of cells in a 160-day-old BM LoMAC culture before (FIG. 15A) and after (FIG. 15B) stimulation with erythropoietin (2 unit/ml) for 16 days. In FIG. 15A, two large megakaryocytes (one mature and one developing) are labeled as “Meg” and two large macrophages were labeled “Mac”. The ploidy of the mature megakaryocyte (with pale cytoplasm) is ˜32N. Please note that none of the macrophages in the LoMAC culture contained inclusion bodies, indicating a total lack of phagocytosis. FIG. 15B shows the culture after stimulation with EPO for 16 days. A very large group of small orthochromic erythroblasts with pyknotic nuclei (in top center) and six basophilic erythroblasts (in left margin) are labeled “EB”. About 50% of all cells in the culture were erythroblasts. Large mature megakaryocytes are labeled “Meg”. The megakaryocyte in the center has ploidy of 32 or higher. About 10-15% of all cells in the culture were megakaryocytes in all stages of differentiation with or without EPO. Wright-Giemsa-stained cytospin preparations. Bars=25 μm.

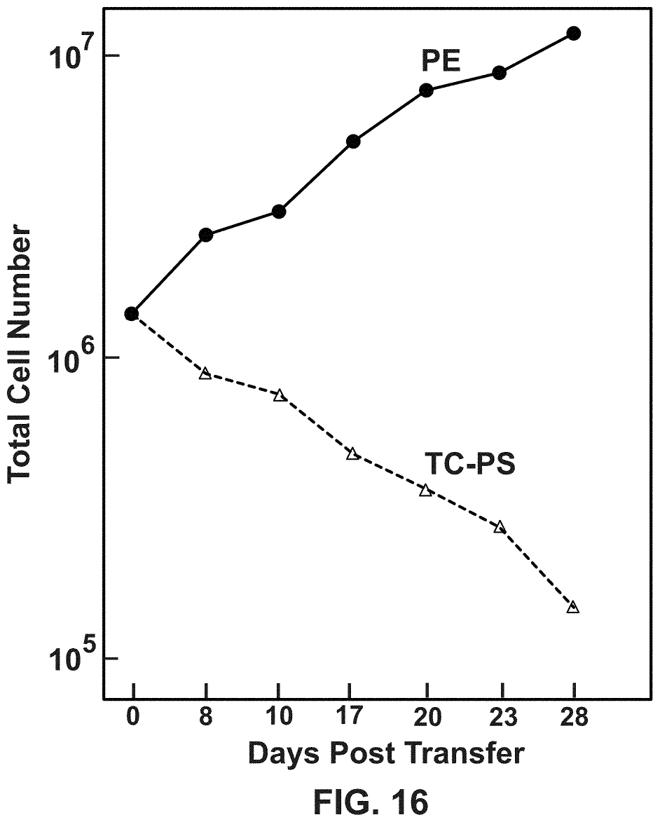

FIG. 16 compares the growth curves of a 260-day-old mouse bone marrow LoMAC culture that was subsequently transferred to TC-PS vs. PE-coated culture devices. This LoMAC culture had undergone 60 subcultivations at a 1:2 ratio by day 260 (i.e. 2⁶⁰-fold expansion). Day 260 bone marrow LoMAC culture contained CAFC_{d35}, B/NK progenitors and most commonly detectable myeloid progenitors. Upon transfer to 6-well plates with the standard TC-PS culture surface, the cell population declined rapidly (broken line) accompanied by the emergence of adherent and non-adherent macrophages. In contrast, cells transferred to a new 6-well plate covered with an LDPE membrane (LoMAC culture) continued to expand as before (solid line). Please note the logarithmic scale on the vertical axis.

FIG. 17 are micrographs of a 80-day-old human BM LoMAC culture showing macrophages alone (FIG. 17A), macrophages plus areas of active hematopoiesis (FIG. 17BB) and macrophages plus numerous small apoptotic bodies in a 100-day-old spent culture (FIG. 17C). Due to the complete absence of phagocytosis, all apoptotic bodies, each representing a dead cell, remained undigested for weeks and months, often forming confluent sheets of dense, dehydrated apoptotic bodies. Please note that many macrophages in FIG. 17C are studded with apoptotic bodies yet there was no evidence phagocytosis on cytospin preparations (not shown). Bars=50 μm.

### REFERENCE NUMERALS

11 The bottom of an LDPE insert that fits inside each well of a matching 6-well cluster plate.
12 The sidewall of an LDPE insert that fits inside each well of a matching 6-well cluster plate.
13 The holed roof of an LDPE insert that fits inside each well of a matching 6-well cluster plate.
14 The opening in the roof of an LDPE insert that fits inside each well of a matching 6-well cluster plate.
15 The bottom of an LDPE insert that fits inside each well of a 6-well cluster plate with a retainer flange at the top.
16 The sidewall of an LDPE insert that fits inside each well of a 6-well cluster plate with a retainer flange at the top.
17 The retainer flange at the top of an LDPE insert that fits inside each well of a 6-well cluster plate.
18 The bottom of one of 6 wells of an LDPE 6-well cluster insert that fits inside a 6-well cluster plate.
19 The flat connecting top of an LDPE 6-well cluster insert.
20 The sidewall of a 60-mm culture dish made of PS with a “super-deep dish” design and an LDPE membrane covering both the bottom and sidewall culture surfaces. Alternatively, the entire dish can be fabricated from transparent, rigid polymers with protein- and cell-binding characteristics similar to those of LDPE such as PMP.
21 The cover of a 60-mm culture dish with a “super-deep dish” design. The cover can be made of any transparent, rigid polymer such as PS.

### DETAILED DESCRIPTION OF THE INVENTION

As used herein, the term “HSC” refers to hematopoietic stem cells with all hematopoietic lineage potentials and long-term repopulating capability in vivo. HSC can be enumerated by day-35 “cobblestone-area-forming cells” (CAFC_{d35}) assay in vitro or by transplantation studies in vivo. The term “cobblestone area” refers to groups or patches of phase-dark blast-like cells that are packed side-by-side and resemble tightly packed cobblestones. Each cobblestone area contains 5 to >10⁵ phase-dark cells. In the data presented in this application, we exclude cobblestone areas that contain fewer than 8 phase-dark cells. Therefore, it is a more stringent criterion. The term “hematopoietic progenitors” refers to progenies of HSC with more restricted lineage potential that can proliferate further before they complete terminal differentiation. “Lymphoid” refer to cells such as B lymphocytes, T lymphocytes, natural killer (NK) cells, NKT cells and their progenitors. “Myeloid”, meaning “of the marrow”, refers to all hematopoietic lineages other than the lymphoid lineage. The term “myeloid” is also used to denote the monocyte/macrophage and neutrophil lineages in certain context to contrast with the “erythroid” lineage. “Granulocyte” includes neutrophil, basophil, mast cell and eosinophil, all of which contain cytoplasmic granules that are neutrophilic, basophilic or eosinophilic. “Erythroblast” refers to nucleated erythroid precursors that have not completed all processes of terminal differentiation such as hemoglobin synthesis or enucleation. “MNC” stands for “mononuclear cells”, which are usually obtained by density-gradient centrifugation of blood, marrow or spleen cell preparations over a step gradient such as NYCODENZ™ and FICOLL-HYPAQUE™ (p=1.077 g/cm³) and have densities lower than 1.077 g/ml. They include HSC, progenitors, monocytes, macrophages and lymphocytes but not mature red blood cells (RBC), neutrophils or other granulocytes, all of which have densities greater than 1.077 g/cm³. “Phagocytes” refers to white cells capable of phagocytosis and include macrophages, monocytes and neutrophils. Macrophages play many roles in normal physiology and pathological states and have the capacity to differentiate into cells with diverse phenotypes depending on the environment. “Activation” or “activation/differentiation” of macrophages usually refers to further change or differentiation in macrophage functions in response to infections or other stimuli that results in production of pro-inflammatory cytokines (e.g. TNF_{α}, interleukin-1 or IL-1, IL-6, IL-12), chemokines (e.g. IL-8, macrophage inhibitory protein-1α or MIP_{1α}, MIP_{1β}) and enzymes (e.g. matrix metalloprotease or MMP). Such macrophages are described as “M1” macrophages or “classically activated” macrophages. Phagocytosis is an important function of M1 macrophages. Certain cytokines and hormones such as hydrocortisone can redirect macrophage differentiation from a pro-inflammatory state toward an “M2” (also know as “anti-inflammatory” or “alternatively activated”) state characterized by decreased production of pro-inflammatory mediators and increased production of factors that promote healing and tissue repair (e.g. arginase, transforming growth factor-β or TGF_{β}, vascular endothelial growth factor-α or VEGF_{α}, fibroblast growth factor or FGF, platelet derived growth factor or PDGF, insulin-like growth factor-1 or IGF-1). (3-5)

The following are commonly used terms and abbreviations for various types of hematopoietic cells in the art. HSC: hematopoietic stem cell. CMLP: common myelo-lymphoid progenitor; CLP: common lymphoid progenitor; CFU-GEMM: colony-forming unit-granulocyte, erythrocyte, macrophage, megakaryocyte; BFU-E: burst-forming unit-erythroid; CFU-EM: colony-forming unit-erythroid, megakaryocyte; CFU-Mk: colony-forming unit-megakaryocyte; CFU-E: colony-forming unit-erythroid; CFU-GM: colony-forming unit-granulocyte, macrophage; CFU-G: colony-forming unit-granulocyte; CFU-M: colony-forming unit-macrophage (or monocyte). The following abbreviations for hematopoietic growth factors are used in this application: KL stands for c-kit-ligand, also known as stem cell factor or steel factor; EPO stands for erythropoietin; TPO stands for thrombopoietin, also known as mpl ligand; GM-CSF stands for granulocyte-macrophage colony-stimulating factor; G-CSF stands for granulocyte colony-stimulating factor; M-CSF stands for macrophage colony-stimulating factor. The total nucleated cells (TNC) count is determined by staining cell suspensions with 0.1% methylene blue (which stains nuclei intensely) in 3% acetic acid. It includes all cells with nuclei and excludes mature red blood cells and platelets. Crude or whole BM refers to total BM cells harvested from bone marrow cavities. BM or peripheral blood (PB) or cord blood (CB) MNC refers to light-density (p<1.077 g/cm³) MNC fractions of BM or CB or PB obtained by density gradient centrifugation over solutions like NYCODENZ™ or FICOLL-HYPAQUE™. Enriched BM, CB or PB refers to BM, CB or PB preparation that has been enriched to various degrees by techniques such as density gradient centrifugation, antibody-based depletion, antibody-based enrichment, magnetic bead separation and fluorescence-activated cell sorting (FACS). ELISA is the abbreviation for “enzyme-linked immunosorbent assay”.

The term “tissue culture device” refers to individual tissue culture dishes (or culture plate), cluster plates with a plurality of wells (e.g. 6-, 12-, 24-, 48-, 96-well, etc.), culture flasks, culture tubes, culture bags and other tissue culture containers such as bioreactors. The term “culture surface” refers to all tissue culture surfaces that are temporarily or permanently in contact with cells or culture media during cultivation or manipulation such as feeding, pipetting and washing. It may include the entire bottom plus sidewalls of a tissue culture device but usually not the cover of a dish or the cap of a flask. In the case of flasks, it includes all internal surfaces that may come in contact with the culture medium or cells in normal operation including the flat top of the flask when positioned horizontally.

The term “hydrocarbons” refers to molecules that contain only hydrogen and carbon atoms. The term “olefin” (literally “oil-forming”), also known as “alkene”, refers to naturally occurring hydrocarbons containing two or more carbon atoms and one or more double bonds between carbon atoms. Olefins are examples of unsaturated hydrocarbons. The simplest olefin is ethylene (H₂C═CH₂), which is a gas found in or derived from petroleum or natural gas. Ethylene can be polymerized via the C═C double bonds to form polyethylene (PE), which is the most produced plastic in the world. PE is an example of “polyolefins”, which also include polypropylene (PP) and poly(4-methyl-1-pentene)(PMP). “Polyolefins” used here refers to polymers of alkenes and copolymers thereof. Polyolefins as a group exhibit many similar characteristics such as high hydrophobicity, chemical resistance and low wettability but can differ substantially in other attributes. PE and PP are not very transparent while PMP is. PE, PP and PMP also have different molding characteristics. Low-density polyethylene (LDPE) has a high degree of short- and long-chain branching with a density range of 0.910-0.940 g/cm³. LDPE has a lower tensile strength but increased ductility. Linear low-density polyethylene (LLDPE) is a relatively linear polymer with fewer short branches and a density range of 0.915-0.925 g/cm³. LLDPE has a higher tensile strength, puncture resistance and transparency than LDPE. High-density polyethylene (HDPE; ρ=0.930-0.970 g/cm³) has little branching and high tensile strength. A widely used non-polyolefin plastic is polystyrene (PS), which is a polymer of styrene. PS consists of long-chain hydrocarbons wherein alternating carbon atoms are linked to the hexagonal phenyl groups (benzene ring) rather than hydrogen atoms as in PE.

Virtually all plastic tissue culture dishes, cluster plates and flasks in use have been fabricated from PS by injection molding and the culture surfaces further treated with ionized oxygen plasma to incorporate many O₂ into PS with the objective of increasing its hydrophilicity and negative charges and hence higher protein-binding capacity, which translates into higher cellular adhesion. Oxygen plasma-treated PS (PS-O2) is often referred to as “tissue culture-treated PS” or TC-PS. The following MATERIALS AND METHODS were used in the examples that follow.

### Preparation of PE-Coated Tissue Culture Devices.

To prepare PE-coated 6-well cluster plates, die-extruded LDPE or LLDPE membranes (100-200-μm thick) were heat-molded with the assistance of vacuum applied through an opening in the dome of the metal mold into individual (FIG. 1&2) or 6-per-cluster (FIG. 3) inserts that match the shape and inner dimensions of individual dishes or the wells of 6-well cluster plates with deep wells. The molding method was essentially the same one that has been in use for the production of sealed air half bubbles of BUBBLE WRAP™ since 1957. Each deep well has a capacity of 16 or more mL and a surface area of about 9.5 cm² at the bottom. The “deep wells” or “super deep dishes” accommodate substantially more culture medium per well or dish in order to provide more nutrients and buffering capacity for large numbers of BM cells, avoid spillage and more importantly, minimize disturbance of BM cells at the bottom during medium exchange. The 6-well base plate is made of either untreated PS or TC-PS. The PS or TC-PS base plate functions only as the housing for the LDPE or LLDPE insert (referred to as the “PE insert” heretofore) and never comes in contact with tissue culture medium or cells. Therefore, it can be substituted with any transparent material such as other hydrocarbon polymers, polycarbonate, acrylic or glass. The PE inserts may remain removable or affixed to the PS or TC-PS base plate by adhesives. The top of the PE insert (FIG. 1-3) may be laminated with PE, PP, PS, polycarbonate or acrylic to increase its rigidity and durability. The resultant combination is referred to as “PE-coated dish” or “PE-coated plate” or “PE-coated well”. The application of a thin layer of PE over the PS- or TC-PS-based dish or well preserves the rigidity and optical clarity of original PS or TC-PS dish or well and allows bright-field or phase-contrast microscopy of cultured cells. It is imperative that the bottom and the entire sidewall (circumference) of each culture dish or well are covered by PE such that the culture media or cells never come in contact with PS or TC-PS in order to prevent accidental adhesion/activation of phagocytes. The PE inserts were sterilized by 70% ethanol in water (vol./vol.) or ethylene oxide gas. The covers of culture plates can be made of PS or any transparent, rigid plastic material since it will not be in contact with the culture medium or cells.

When preparing individual PE-coated culture dishes, the base dishes should have a “super deep dish” design as illustrated in in FIG. 4. The ideal ratio of dish height to bottom diameter of deep dishes will vary with the diameter but in general must be >0.2 at the minimum. In the examples in FIG. 4, the height-to-diameter ratios for 35-, 60- and 100-mm dishes are 0.51, 0.42 and 0.32, respectively. The “super deep dish” design is a functionally important feature that allows the use of more culture medium (compared with traditional tissue culture dishes) in order to provide sufficient nutrients and buffering capacity for large numbers of BM cells, avoid spillage and contamination, and minimize disturbance to cells at the bottom during frequent medium exchange and very long periods (months) of cultivation. The cover of individual super deep dish can be fabricated from any transparent, rigid plastic material.

### PMP Tissue Culture Devices.

Poly(4-methyl-1-pentene), abbreviated as PMP, is a polyolefin with PE-like property such as hydrophobicity and low protein/cell binding but higher transparency, melting point (240° C.) and shape retention. PMP culture dishes or other formats of tissue culture wares were fabricated from commercially available virgin PMP resin using the standard injection molding technique and sterilized by ethylene oxide gas.

Comparison of Fibroblast Adhesion in PS vs. TC-PS vs. PE-Coated Plates.

The immortalized fibroblastoid bone marrow stromal cell line OP-9 (20) was maintained in Dulbecco's Modified Eagles' medium (DME with 4.5 g/dL glucose; Gibco) supplemented with 10% (vol./vol.) fetal bovine serum (FBS; Hyclone), 1 mM L-glutamine (Gibco), penicillin (100 I.U./mL) and streptomycin (100 μg/mL; Gibco). OP-9 is completely anchorage-dependent. To compare the adhesion of OP-9 to different culture surface, logarithmically growing OP-9 cells were washed with phosphate-buffered saline (PBS; pH 7.4) and detached by trypsin/EDTA (Gibco), counted and seeded in triplicates at 4.0×10⁵ cells per well in traditional 6-well PS plates that had not been tissue culture-treated or in 6-well PS plates that had been tissue culture-treated (TC-PS) or in PE-coated plates. Cells were fed with the growth medium and incubated at 37° C. in a water-jacketed incubator equilibrated with 5% CO₂ and 95% air. After 24 hr., non-adherent cells were removed by gentle rinsing with phosphate-buffered saline. Adherent cells were trypsinized, neutralized with growth medium, mixed with trypan blue and counted using a hemocytometer method.

Comparison of Macrophage Adhesion in PS vs. TC-PS vs. PE-Coated Plates.

The WEHI-3B cell line is a mouse myelomonocytic cell line that exhibits many properties of macrophages such as phagocytosis and secretion of macrophage cytokines (21). It is used as surrogate macrophages in certain studies (22). WEHI 3B may grow as a suspension culture but most cells adhere quickly to PS or TC-PS culture surface. It was maintained in the same medium as for OP-9. To compare the adhesion of WEHI-3B cells to different culture plates, non-adherent WEHI-3B cells of a logarithmically growing culture were seeded in triplicates at 3.0×10⁵ cells per well in PS or TC-PS or PE-coated 6-well plates. Cultures were incubated at 37° C. in a water-jacketed incubator equilibrated with 5% CO₂/95% air. After 24 hr., nonadherent cells were removed and the culture rinsed gently with 37° C. PBS. Adherent cells were trypsinized, washed with growth medium and counted a hemocytomer.

### Mouse BM LoMAC Culture in PE-Coated Culture Devices.

The basic long-term bone marrow culture medium consisted of a 1:1 mixture of Rosewell Park Memorial Institute 1640 (RPMI 1640; Gibco) and Iscove's Modified Dulbecco's Medium (IMDM; Gibco) supplemented with 20% (vol./vol.) donor horse serum (HS; Gibco), 1 mM L-glutamine, penicillin (100 I.U./ml), streptomycin (100 μg/ml), 2-mercaptoethanol (2-ME; 5×10⁻⁵ M; Sigma) and sodium hydrocortisone hemisuccinate (HC; 1×10⁻⁶ M; Sigma). Basic long-term culture medium was supplemented with 40 ng/ml mouse c-kit-ligand (mKL; PeproTech)(23-26) or 4-5% (vol./vol.) of the conditioned medium of BHK/KL as a source of mKL and 40 ng/ml mouse thrombopoietin (mTPO; PeproTech)(27) or 4-5% (vol./vol.) of the conditioned medium of BHK/TPO as a source of mTPO. Addition of mKL was necessary as KL is essential for the survival by HSC and the main source of KL is the fibroblastoid stromal cells (23-26), which were absent in BM LoMAC culture due to complete lack of fibroblast adhesion to the culture surface. The BM of the tibias and femurs of 8 to 20-week-old C57BL/6 mice were flushed out using a syringe fitted with a 25-guage needle. The harvested BM were pooled and pipetted gently to create a more even suspension of marrow particles. Marrow particles were preserved as much as possible. The equivalent of approximately 60-70% of the bone marrow cells from “one bone” (averaged tibia/femur) of a 12 to 20-week-old adult mouse or ˜2×10⁷ total nucleated cells (TNC) were cultured in 6 ml of long-term bone marrow culture medium per well (TC-PS vs. PE-coated) in a 6-well plate. When younger mice (<8 weeks) were used, the marrows of two bones were combined in one well of a PE-coated 6-well cluster plate to provide a more suitable starting cell dose. Cultures were incubated at 37° C. (instead of 33° C. as in traditional LTBMC) in a water-jacketed incubator equilibrated with 5% CO₂ and 95% air. One half to one third of the medium was replaced every 2-3 days with minimal disturbance of the cells at the bottom. When a BM LoMAC culture became overcrowded, the culture was re-suspended by moderate pipetting and subdivided into two wells, including the old well. The old well was reused as it usually contained some adherent HSC. Surplus cells may be cryopreserved in growth medium supplemented with 10% (vol./vol.) dimethylsulfoxide (DMSO; Sigma). Experience indicates that it is advisable not to divide BM LoMAC cultures at a splitting ratio greater than 1:2 or more often than once every 5-10 days. The control cultures were established in TC-PS (and PS in some experiments) 6-well plates and processed in parallel. To avoid accidental activation of macrophages, all procedures were performed using PP instead of PS test tubes.

### Day 35 Cobblestone-Area-Forming Cell (CAFC_{d35}) Assay.

The standard CAFC_{d28} assay is the considered the best in vitro assay for mouse HSC (28-30). Its value corresponds to or correlates with the number of long-term (>4 months)-repopulating HSC in bone marrow transplant studies (28, 29). In our studies, a more stringent CAFC_{d35} assay at 37° C. was used. It was performed essentially as described by Ploemacher et al with slight modifications (28, 29). The fibroblastoid stromal cell line OP-9 (20) instead of primary bone marrow mixed macrophage-fibroblast stroma was used since the clonal OP-9 provided a more uniform and consistent environment. Another advantage is that OP-9 does not produce macrophage colony-stimulating factor (M-CSF) that complicates the analyses. Due to the high concentrations of hematopoietic progenitors in a typical BM LoMAC culture, 24-well plates instead of 96-well plates were used for CAFC assay to ensure the stromal cell areas were not overcrowded with or even destroyed by hematopoietic progenitors or otherwise limiting. To prevent detachment of OP-9 cells during the long assay period, the TC-PS 24-well plates were pre-coated with 0.5% gelatin in water (wt./vol.) for 2 hr. and air-dried before use. OP-9 stromal cell line was seeded in gelatin-coated 24-well plates 3-4 days before CAFC assay and allowed to grow to confluence. The OP-9 monolayers were then treated with mitomycin C (Sigma) at 5-8 μg/ml for an hour and washed with PBS twice before addition of hematopoietic cells. At least four different progenitor cell doses (dilutions) and 6-12 wells per cell dose were used in each CAFC_{d35} assay. The culture medium for CAFC_{d35} assay consisted of a 1:1 mixture of RPMI 1640 and IMDM supplemented with 15% (vol./vol.) HS, 1 mM L-glutamine, penicillin/streptomycin, 2-ME (5×10⁻⁵ M) and HC (1×10⁻⁶ M). Cultures were incubated at 37° C. instead of 33° C. (28, 29) in a water-jacketed incubator equilibrated with 5% CO₂ and 95% air. One third of the medium was replaced every 2-3 days or when the medium was too acidic (pH<7.0). CAFC scoring was performed on day 35 of the assay instead of day 28 as in standard CAFC assay (28, 29) using an inverted microscope equipped with phase contrast and a 20× objective. Wells that contain at least one cobble stone area consisting of 8 (instead of 5 as in standard CAFC_{d28}) or more stroma-embedded, tightly packed, phase-dense blast cells were scored as positive (28). In most positive wells, the cobblestone areas contained hundred to thousands of cells. The number of CAFC_{d35} was calculated according to the method of maximal likelihood in accordance with Poisson distribution principle (28-30). Please note that our CAFC_{d35} method was more stringent than the standard CAFC_{d28} method for three reasons: (i) incubation was at 37° C. instead of 33° C. and (ii) a minimum of 8 closely packed cells instead of 5 was required to qualify as a cobblestone area and (iii) scoring of CAFC was performed on day 35 instead of day 28.

### Assays of Erythroid, Myeloid and Lymphoid Differentiation.

BM LoMAC cultures typically contained monocytes/macrophages, neutrophils, megakaryocytes and small numbers of basophilic erythroblasts in addition to partially differentiated progenitors and undifferentiated blasts of various lineages. To evaluate the frequencies of various hematopoietic progenitors in colony-forming-cell assays in semi-solid medium, aliquots of LoMAC cultures were plated in 0.8% methylcellulose in IMDM supplemented with 20% FBS, bovine serum albumin (BSA; Sigma), penicillin-streptomycin, 2-ME (5×10⁻⁵ M), mKL (10 ng/ml), mTPO (10 ng/ml), mIL-3 (5 ng/ml; PeproTech) and mouse granulocyte-macrophage colony-stimulating factor (mGM-CSF; 5 ng/ml; PeproTech) and incubated in humidity chambers at 37° C. in 5% CO₂ and 95% air for 14-16 days. Colonies were scored on days 10-16.

To investigate the lymphoid potential of cells harvested from mouse BM LoMAC cultures, aliquots of cells were co-cultured with monolayers of mitomycin C-treated OP-9 cells in gelatin-coated 24-well plates in a 1:1 mixture of DME and RPMI supplemented with 5% FBS, penicillin-streptomycin, 5×10⁻⁵ M 2-ME, human Flt3 ligand (hFlt3L; 2 ng/ml; R&D) and mouse interleukin-7 (mIL-7; 2 ng/ml; R&D) for 14-21 days to support the development of early lymphoid progenitors as well as myeloid progenitors. OP-9 supplied membrane-form mKL in these co-cultures. After 14-21 days, cultures were stimulated with mIL-7 alone at a higher concentration (5-10 ng/ml) to support the further development of committed B and NK progenitors/precursors. When the OP-9 cells (source of membrane form mKL) were destroyed after the development of functional NK cells, which were cytotoxic against OP-9 (31; U.S. Pat. No. 9,121,008), cultures were supplemented with soluble mKL (5 ng/ml) in addition to mIL-7 (5-10 ng/ml). Aliquots of cells were obtained periodically and stained with B220-FITC (BioLegend) plus CD19-PE (BioLegend) or CD3-FITC (BioLegend) plus NK1.1 (Pharmingen) monoclonal antibodies (mAb) to detect pre-pro B, pro B, pre B, T and NK cells. The hematopoietic nature of cells was confirmed by co-staining with directly labeled CD45.2 mAb (BioLegend). Cells were also examined microscopically using cytospin preparations stained with the Wright-Giemsa stain. To support complete erythroid differentiation in liquid culture, BM LoMAC cultures were supplemented with human EPO (hEPO; 1-2 unit/ml; Amgen) for 12 to 16 days.

### Immunofluorescence Detection of Cell Surface Lineage Markers.

Cultured cells were washed with Hanks' buffered salt solution (HBSS) supplemented with 5% of FBS and 0.009% sodium azide (HSFAH) and incubated with FcBlock (anti-CD16/CD32; 0.125 μg per 10⁵ cells)(BioLegend) for twenty minutes and then stained for 40 min. with direct conjugates of CD45.2, B220, CD19, CD3, NK1.1, CD41 and additional mAb and washed with HSFAH twice before flow cytometry or immunofluorescence microscopy.

### Human LoMAC Cultures in PE-Coated Plates.

Human BM LoMAC culture was performed essentially as for mouse BM LoMAC with some differences. Instead of the whole BM, commercially available or archived, cryopreserved MNC of BM from healthy donors with no identifiers were used. The conditioned media of BHK/KL, BHK/TPO and J558L7 were used in some experiments in lieu of purified human factors as mKL , mTPO and mIL-7 are all active on human cells. Human cord blood LoMAC culture was performed essentially as for human BM LoMAC culture using commercially available or archived cryopreserved MNC of cord blood from umbilical cords with no identifiers. Human PBSC LoMAC was performed essentially as for human BM LoMAC culture using commercially available or archived cryopreserved MNC of peripheral blood from healthy donors with no identifiers.

### Embodiments

### EXAMPLE 1

Fibroblasts and Macrophages Exhibit Very Low Adherence to PE Compared with PS or TC-PS.

To compare the adhesion of fibroblasts to different culture surfaces, we seeded equal numbers of OP-9 fibroblastoid stromal cells in PS vs. TC-PS vs. PE-coated 6-well plates. After 24 hours of incubation at 37° C., adherent cells were trypsinized and counted. As shown in FIG. 5, OP-9 adhered very efficiently to both PS and TC-PS surfaces with >100% of input cells found in the adherent fraction as some mitoses had taken place during the incubation period. In contrast, very few OP-9 cells bound to the PE-coated plates with an efficiency of <1% of that of PS or TC-PS. A parallel study was performed using a macrophage-like cell line, WEHI 3B. Again, WEHI 3B adhered efficiently to both PS and TC-PS surfaces but only marginally to the PE culture surface with an efficiency of 2-3% of that or PS or TC-PS (FIG. 6). These results were in line with the prediction based on the differences in protein-binding capacities of PS, TC-PS and PE.

Interestingly, the macrophage like cell line WEHI 3B grown in PE-coated plates were able to down shift cell cycling and metabolism and enter a quiescent stage once they reached a certain population density and thereby prevented the culture media from becoming acidic even though no fresh medium was added or exchanged during the entire experimental period (21 days)(FIG. 7, solid line). In contrast, WEHI 3B grown in TC-PS plates grew rapidly and continued to cycle and metabolize at a high rate after reaching a peak cell density. As a result, the culture media became acidic (pH<6.7) quickly, followed by cell death due to depletion of nutrients and acidity (FIG. 7, broken line). These observations indicate that the adhesion (e.g. via integrin receptors) of WEHI 3B cells to TC-PS provided such strong mitotic and/or functional stimuli that they were unable to down shift and exit the cell cycle when nutrients were becoming depleted. In contrast, WEHI 3B grown in PE-coated plates were able to slow down and even become quiescent, perhaps due lack of cellular adhesion to the culture surface. Similar findings were made with primary mouse BM macrophages (not shown).

The ability of PE-coated plates to prevent the macrophage-like cell line WEHI 3B from going into overdrive proved to be a very useful property in BM LoMAC cultures. During long-term culture of BM in PE-coated culture devices, the culture medium rarely became acidic even if no fresh medium was added for 5-10 days and the cells were able to enter a quiescent state. This is very different from traditional LTBMC, which is in constant danger of nutrient depletion and high acidity and therefore requires careful monitoring and timely medium exchange. All evidence points to macrophages as the main culprit.

### EXAMPLE 2

### LoMAC BM Long-Term Culture Using PE-Coated Culture Devices.

To test the hypothesis that macrophages in traditional long-term BM cultures might be harmful to HSC and progenitors, we compared mouse BM long-term cultures in TC-PS vs. PE-coated plates. The assumption was that reduced macrophage adhesion in PE-coated plates would result in less macrophage M1 activation, which in turn would help create a non-inflammatory environment or an anti-inflammatory environment if HC is also present. BM cultures were incubated at the physiologic 37° C. instead of 33° C. as required in traditional LTBMC. In line with the findings using the OP-9 stromal cell line (FIG. 5), PE completely prevented the adherence of fibroblastoid BM stromal cells, which underwent apoptosis without anchorage. As a result, there were no fibroblastoid stromal cells in BM cultures set up in the PE-coated culture devices. However, there were small numbers of adherent macrophages, most of which adhered only loosely to the PE surface (see FIG. 9B below). These macrophages were rounded in shape and did not display the well-spread morphology typical of adherent macrophages in BM cultures established in TC-PS culture devices (see FIG. 9A below). Overall, the adherence of macrophages was greatly reduced compared with BM cultures set up in TC-PS wells. Furthermore, no foreign body giant cells (FBGC) were seen in BM cultures established in PE-coated culture devices while they were numerous in BM cultures set up in TC-PS culture devices (see FIG. 9A).

As fibroblastoid stromal cells are the main source of KL and HSC and most hematopoietic progenitors depend on KL for survival (19-22), exogenous KL must be provided in BM cultures performed in PE-coated tissue culture dishes. Therefore, we added recombinant mKL (40 ng/ml) or 4-5% (vol./vol.) BHK/KL-conditioned medium to all mouse BM cultures set up in PE-coated plates. While KL alone could support long-term hematopoiesis in cultures set up in PE-coated dishes to some extent, the addition of recombinant mTPO (40 ng/ml) or 4-5% (vol./vol.) BHK/TPO-conditioned medium (a source of mouse TPO) significantly improved the performance of such cultures and greatly increased megakaryocyte production. This is not a surprise since HSC express both c-kit and c-mpl receptors (for KL and TPO, respectively) and KL or TPO can independently stimulate the proliferation of HSC as well as many hematopoietic progenitors (32, 33). Control cultures were set up in traditional TC-PS plates and fed with the same culture medium containing HC (10⁻⁶ M), mKL and mTPO and processed in parallel.

FIG. 8 compares the growth curves of BM cultures using PE-coated vs. TC-PS culture plates. While the culture in the TC-PS plate declined after day 20-24 and became spent after day 36-48, the parallel culture in PE-coated plate continued to expand in a quasi-logarithmic way past day 40 and beyond. While all mouse BM cultures established in PE-coated plates continued to expand beyond day 300 whenever such attempts were made, they were usually cryopreserved after day 120. The results of BM cultures set up in untreated PS plates (not shown) were similar to those using TC-PS plates. The contrasting results between cultures established in TC-PS vs. PE-coated culture devices suggest that inhibitors or toxins of hematopoiesis are produced in BM cultures set up in TC-PS (or PS) culture devices and this is one of the reasons why it has been so difficult to maintain HSC in vitro. The use of PE-coated plates overcomes this obstacle.

FIG. 9 compares the appearance of mouse BM cultures established in TC-PS vs. PE-coated plates. FIG. 9A is a phase-contrast micrograph of the control culture established in a TC-PS well on day 18 of the culture. “Mac” labels adherent macrophages. “FBGC” indicates a foreign body giant cell. Small-to-medium round refractile cells are progenitors, monocytes and neutrophils. FIG. 9B is a phase-contrast micrograph of a day 18 mouse BM culture established in a PE-coated well. “Meg” marks megakaryocytes, which are numerous in BM cultures established in PE-coated culture devices and few in control cultures established in TC-PS wells. “Mac” labels floating macrophages. The remaining small-to-medium round, refractile cells were monocytes, neutrophils and progenitors. Adherent, well-spread macrophages or FBGC that dominated BM cultures set up in TC-PS plates were very rare in BM cultures set up in PE-coated culture devices.

### EXAMPLE 3

Comparison of Phagocytic Activity and TNF_{α} Production in Mouse Bone Marrow Cultured in PE- vs. PS-Coated Plates.

In addition to reduced adhesion, macrophages (pre-existing and newly generated) in BM cultures established in PE-coated culture devices were less phagocytic (as evidenced by lower frequencies of inclusion bodies) in contrast to macrophages found in BM cultures set up in TC-PS culture devices (FIG. 10). In fact, de novo generated macrophages like those in older, well-established BM cultures in PE-coated culture devices were completely non-phagocytic, with none of the macrophages containing inclusion bodies (see FIG. 15A and FIG. 17C below).

To compare the effects of PE on the production of pro-inflammatory cytokines (as an indicator of macrophage pro-inflammatory activation), we set up bone marrow cultures in TC-PS-based dishes vs. PE-coated dishes in the absence or presence of HC (10⁻⁶ M). As predicated, HC reduced the production of the key pro-inflammatory and pro-apoptotic cytokine tumor necrosis factor-α (TNF_{α}) in cultures established in both TC-PS- and PE-coated plates. The production of pro-inflammatory cytokines was further reduced in cultures set up in PE-coated dishes (FIG. 11). Other key pro-inflammatory cytokines secreted by M1 activated macrophages such as IL-1 follow a similar pattern (not shown). Although the concentrations of inflammatory cytokines depend on the number of cells and the duration of incubation, the very low concentrations of TNF_{α} (2.5-5.0 μg/ml; FIG. 11, solid black bars) detected in BM cultures using PE-coated devices and in presence of HC put the macrophages in these cultures firmly in the non-inflammatory (or anti-inflammatory) category. The decreased phagocytosis in day 12 mouse BM culture (containing preexisting BM macrophages) using PE-coated devices (FIG. 10) and the complete lack of phagocytosis on day 160 (containing mostly de novo generated macrophages)(FIG. 15A) suggest that these macrophages are in a profound non-inflammatory state. This interpretation is further supported by the complete absence of phagocytosis by macrophages in the day 90 human BM culture in PE-coated devices, where the complete absence of phagocytosis by macrophages manifested itself in the buildup of a sea of apoptotic cell bodies—a phenomenon never reported before (FIG. 17C below).

### EXAMPLE 4

Numbers of HSC in BM Cultures Established in TC-PS vs. PE-Coated Plates.

The numbers of HSC present in the starting BM inoculums and on day 12, 24 and 36 of bone marrow long-term cultures were determined by CAFC_{d35} assay performed at 37° C., which is more stringent than the standard CAFC_{d28} assay performed at 33° C. For brevity, we will refer to the BM cultures established in PE-coated devices as LoMAC (Low-Macrophage-Adhesion/Activation) cultures heretofore. As shown in FIG. 12 (solid line), the number of CAFC_{d35} was 262 per LoMAC culture (started with cells harvested from one leg bone on average), in line with the reported number of HSC per leg bone as determined by competitive repopulation BM transplantation (34, 35). The number of CAFC_{d35} increased to 579 on day 12 per LoMAC culture. CAFC_{d35} was 300 on day 24 and 305 per LoMAC culture on day 36 (FIG. 10, solid line). In contrast, CAFC_{d35} declined very rapidly in BM cultures set up in regular TC-PS wells and was undetectable after day 12 (FIG. 12, broken line). Similarly rapid decline of CAFC_{d35} was seen in BM cultures set up in untreated PS (Petri dishes; FALCON®) or “Ultra Low Adhesion” plates (ULA plates; CORNING®) or super low binding plates (PlusS plates; Alpha Plus Scientific Corporation)(not shown).

The cultures described in the preceding section (FIG. 12) were started with 1.0×10⁷ (TNC) BM cells per well of a 6-well cluster plate and the cultures were pipetted and sampled very frequently (every 2-3 days) in order to establish the growth curves and perform CAFC assays. When larger numbers of BM cells (e.g. 2.2×10⁷ BM cells per well of a 6-well cluster plate) were used to initiate the cultures and pipetting and sampling were minimized, BM cultures grew more robustly and HSC were expanded to a greater extent as shown in FIG. 13 (solid line). In this example, HSC had expanded by at least 40 folds by day 56. This is a dramatic improvement over the culture established in TC-PS plates in which no HSC could be detected after day 28 (FIG. 13, broken line). The inference of the positive effect of higher starting cell number on the performance of BM LoMAC cultures is that there must be some kind of paracrine stimulation in BM LoMAC cultures. We also noticed that more HSC remained within the original well than in the new well when the cultures were divided, suggesting that some HSC adhered to the PE-coated culture surface. Furthermore, frequent manipulation of the culture such as pipetting and sampling resulted in a more rapid decline of HSC, suggesting that adhesion to the PE culture surface or other cells or cell aggregates in BM LoMAC culture conferred some survival or growth advantage on HSC.

FIG. 14 compares the numbers of CAFC_{d35} in BM cultures started with day 5 post-fluorouracil bone marrow from a 12-week-old C57BL/6 mouse (as in FIG. 12) on day 0, 12, 24 and 36. The number of CAFC_{d35} in FIG. 14 is for cultures that were set up using day 5 post-5-fluorouracil marrow harvested from the equivalent of one leg bone as in FIGS. 12&13. 5-fluorouracil selectively kills cells that are rapidly cycling. Thus, the day 5 post-5-fluorouracil bone marrow contained mostly quiescent HSC, very slowly cycling primitive hematopoietic progenitors and some mature cell and provided relevant information on how semi-purified or enriched bone marrow cells might behave in LoMAC cultures. As shown in FIG. 14, HSC again disappeared rapidly from the culture established in TC-PS culture devices (FIG. 14, broken line). In contrast, CAFC_{d35} in cultures set up in PE-coated culture devices not only survived but also increased over time (FIG. 14, solid line).

### EXAMPLE 5

### Lineage Potential of Hematopoietic Progenitors in Mouse BM LoMAC Culture.

Mouse BM LoMAC cultures spontaneously and continuously produce several types of mature hematopoietic cells as well as partially differentiated precursors. These included monocytes/macrophages, segmented and band-form neutrophils, monoblasts, promyelocytes, myeloblasts, megakaryoblasts, basophilic erythroblasts and occasional eosinophils, basophils and mast cells. Addition of hEPO at 1-5 unit/ml to mouse BM LoMAC cultures for at least 8-10 days allowed full differentiation of erythroid progenitors. Production of erythroblasts continued as long EPO was present. After 16 days of continuous EPO stimulation, about 50% of all cells in the culture were erythroblasts, which were never seen in traditional LTBMC. In addition, various hematopoietic progenitors were easily detected in colony assays in semi-solid medium in the presence of KL, TPO, IL-3 and EPO at the following frequencies when assayed on day 160 of a representative mouse BM LoMAC culture:
(No. per 1.45×10⁴ cells plated)
6.7 CFU-GEMM,
6.3 CFU-EMeg (bipotent erythroid/megakaryocyte),
11.3 BFU-E,
112.0 CFU-GM,
9.7 CFU-Meg,
36.7 CFU-G,
280.0 CFU-M.

In summary, besides CAFC_{d35}, mouse BM LoMAC cultures generated most myelo-erythroid progenitors detectable by colony assays in semi-solid medium and at frequencies similar to those of moderately enriched bone marrow. Furthermore, certain colonies such as CFU-GEMM, CFU-EM and CFU-Mk colonies were more robust than those formed by freshly harvested bone marrow.

### EXAMPLE 6

### De Novo Erythropoiesis and Megakaryocytopoiesis in the Liquid Phase of BM LoMAC Culture.

A perplexing phenomenon in traditional LTBMC is the complete absence of erythroblasts even in the presence of EPO. De novo erythroid differentiation is limited to the production of a small number of BFU-E, if any at all. In most cases, there is no detectable BFU-E in LTBMC after the first 2-4 weeks. The cause of this failure was unknown. In contrast, BM LoMAC established in PE-coated wells produced not only large numbers of megakaryocytes (FIG. 15A), BFU-E and BFU-EMeg but also numerous erythroblasts at all stages of differentiation when stimulated with EPO (1-2 unit/ml) for 10-16 days (FIG. 15B). As it took at least 8-10 days for the first hemoglobinized erythroblast to appear in mouse BM LoMAC cultures, we concluded that the most mature erythroid progenitor in the mouse BM LoMAC cultures prior to the addition of EPO must be BFU-E.

FIG. 15B is a Wright-Giemsa-stained cytospin preparation of a 160-days-old mouse BM LoMAC culture stimulated with EPO for 16 days. The control culture without EPO stimulation is shown in FIG. 15A. As shown in FIG. 15B, basophilic, polychromatophilic and orthochromic erythroblasts appeared as large colonies consisting of hundreds to thousands of erythroblasts right in the liquid phase (i.e. not colony assay) of BM LoMAC cultures 16 days after the addition of EPO (1-2 unit/ml). After 16 days of continuous exposure to EPO, about 50% of the cells were erythroblasts (FIG. 15B). As it took 8-10 or more days of EPO stimulation for the first erythroblasts clusters to appear in BM LoMAC cultures, we concluded that these erythroblasts were produced from very primitive erythroid progenitors such as BFU-E, CFU-EMeg or CFU-GEMM. The EPO pulses could be applied repeatedly without compromising mouse BM LoMAC cultures.

In addition to large numbers of erythroblasts, numerous megakaryocytes at all stages of differentiation were present in BM LoMAC cultures. Like erythroblasts and erythroid progenitors, megakaryocytes and their progenitors (CFU-Meg or CFU-EMeg) are undetectable or barely detectable in traditional LTBMC. In contrast, megakaryocytes at all stages of differentiation (with nuclear ploidy up to 32N) and their progenitors were produced in BM LoMAC cultures before (FIG. 15A) and after (FIG. 15B) EPO stimulation. Obviously TPO was the primary stimulus while EPO provided synergistic activity for megakaryocytic development.

Since BFU-E, CFU-Meg, CFU-EMeg and CFU-GEMM are short-lived progenitors unless rescued by essential growth factors (e.g. EPO, TPO, GM-CSF), they must be continuously produced from the HSC (or CMLP) in mouse BM LoMAC cultures in order to be detected in colony assays or in liquid cultures. As erythroblasts, megakaryocytes, BFU-E, CFU-Meg, CFU-EMeg and CFU-GEMM were easily detected in mouse BM LoMAC cultures but are virtually undetectable in traditional LTBMC (with added EPO and TPO), our results strongly suggest that this reversal stemmed from a switch from a pro-inflammatory environment in LTBMC to a non-inflammatory or anti-inflammatory one in LoMAC cultures. This has important implications in designing future culture systems for expanding HSC and erythroid and megakaryocytic progenitors.

### EXAMPLE 7

Mouse BM LoMAC Cultures Contained Primitive Progenitors that Could Give Rise to Lymphocytes De Novo.

BM LoMAC cultures did not spontaneously generate morphologically recognizable or marker-B, NK or T cells. The presence of high concentrations of HC killed all preexisting lymphocytes and precluded the generation of new ones. However, large numbers of pre-pro-B, pro-B and NK cells could be generated from BM LoMAC in a three-stage assay. In the first stage, cells from BM LoMAC cultures were washed free of HC and co-cultured with OP-9 stromal cells in the presence of low concentrations of KL, Flt3L and IL-7 for 14-20 days during which committed lymphoid progenitors were generated. The OP-9 co-cultures were then supplemented with IL-7 at a higher concentration for another 10-20 or more days to support the next stage development of committed B and NK progenitors. Large numbers of KL/IL-7-responsive B220⁺CD19⁻ pre-pro B cells were produced continuously for weeks, followed by the appearance of B220⁺CD19⁺ pro B cells. After the appearance of large numbers of mature NK1.1+ NK cells (along with pre-pro B cells), the OP-9 stromal layer was destroyed (lysed) by NK cells as reported before (31). Thereafter the NK cultures were supplemented with KL, IL-7 and IL-2 to support further differentiation of NK cells to the CD3⁻NK1.1⁺ stage as described before (31). Continuous NK cell lines similar to the KIL C.2 cell line (31) could be derived from in vitro generated NK cells.

Taking together the data of CAFC_{d35} assays, liquid cultures, colony assays in semi-solid medium and three-stage B/NK assays, we conclude that mouse BM LoMAC cultures contained hematopoietic stem cells, CMLP, CLP, CFU-GEMM, CFU-EM, BFU-E, CFU-Meg, CFU-E, CFU-GM, CFU-G and CFU-M. Other hematopoietic lineages such as mast cells, basophils and eosinophils were present in small numbers in mouse BM LoMAC cultures (not shown).

Although large numbers of macrophages were present in BM cultures established in both TC-PS- and PE-coated plates, those in PE-coated plates (i.e. LoMAC cultures) contained few or no inclusion bodies (cellular debris)(FIG. 15A), suggesting they were non-inflammatory in nature.

### EXAMPLE 8

### TC-PS Strongly Inhibited Previously Established Mouse BM LoMAC Cultures.

When cells from a 260-day-old mouse BM LoMAC culture were transferred to a TC-PS-based culture plate, the culture declined rapidly as shown in FIG. 16 (broken line). The dramatic decline was accompanied by increasing numbers of adherent and non-adherent macrophages, including FBGC. In contrast, cells transferred to a new PE-coated plate continued to proliferate as before (FIG. 16, solid line). The differing outcomes highlighted the role played by TC-PS in the decline of hematopoiesis in traditional LTBMC.

### EXAMPLE 9

### Human BM and CB LoMAC.

We have also performed LoMAC cultures using cryopreserved human BM MNC (instead of fresh whole BM of mice). The culture medium were the same as for mouse BM LoMAC cultures but higher concentrations of KL and TPO were used (recombinant hKL at 50 ng/ml and hTPO at 80 ng/ml). Alternatively, the conditioned media of BHK/KL (5% vol./vol.) and BHK/TPO (8% vol./vol.) were used as mKL and mTPO cross-reacted with human cells. Under these conditions, human BM MNC proliferated as in mouse BM LoMAC cultures with the major differences being: (i) human megakaryocyte development was blocked at an early stage with most megakaryocytes appearing as micromegakaryocytes containing only one nucleus (2N ploidy) with occasional cells containing 2 nuclei (4N ploidy). The addition of EPO (4 unit/ml) was able to drive more micromegakaryocytes toward 4N ploidy. However, well-differentiated megakaryocytes were rarely seen in human BM LoMAC cultures beyond day 30. This observation indicated that human megakaryocyte development required additional factors for complete development; (ii) a similar differentiation block was observed in erythroid differentiation in human BM LoMAC cultures. The majority of human BFU-E and/or CFU-E were not able to proliferate in the LoMAC environment in response to KL, TPO and EPO (4-5 unit/ml). Instead, they differentiate directly into single, large (˜12-15 μm) poorly hemoglobinized erythroblasts without the proliferation that accompanied normal erythroid terminal differentiation. Thus, human erythroid development in vitro required additional mitogenic factors compared with mouse erythroid progenitors. Addition of hFlt3L (2-6 ng/ml) improved the proliferation and longevity of human BM LoMAC cultures. However, hFlt3L also significantly increases the production of macrophages and dendritic cells, whose impact is undoubtedly complex and requires further investigation.

FIG. 17A is a phase-contrast micrograph of an area of a day 80 human BM LoMAC showing the typical appearance of macrophages, most of which were nonadherent with a tendency to cluster. A small number of macrophages adhered weakly to the PE membrane and were easily detached by pipetting. FIG. 17B shows an area of active hematopoiesis in the same culture. Most small round cells in FIG. 17B were monocytes, neutrophils and progenitors at all stages of differentiation. A cluster of large macrophages was seen in the left margin.

A very unusual feature of human BM LoMAC cultures is the gradual buildup of tens of millions of apoptotic bodies measuring 1-2 μm in diameter (FIG. 17C). This phenomenon has never been observed before. This finding suggests that macrophages that developed in BM LoMAC cultures were non-phagocytic. This is supported by the complete lack of inclusion bodies in macrophages from well-established human BM or CB or PBSC LoMAC cultures in cytospin preparations (not shown). This is consistent with the findings in mouse BM LoMAC cultures (FIGS. 10&15A).

### EXAMPLE 10

LoMAC Cultures in Tissue Culture Devices Fabricated from poly(4-methyl-1-pentene)(PMP).

Polyolefins such as PE, PP and PMP share similar characteristics such as high hydrophobicity, low protein binding, low cell binding and high resistance to chemicals. However, they differ in many other ways such as transparency, melting temperature, tensile strength, shape retention and molding characteristics. Due to its low protein binding, high transparency and rigidity, PMP is a good candidate for making tissue culture devices that might offer the same benefits as PE-coated culture devices. Therefore, we tested tissue culture devices fabricated from PMP for their ability to support long-term hematopoiesis in LoMAC cultures. The results indicated that PMP tissue culture devices provided better support for long-term hematopoiesis than TC-PC culture devices, yielding results very similar to those shown in FIGS. 5-17 using PE-coated culture devices.

### ADVANTAGES OVER PRIOR ART

### Distinct Advantages of PE-Coated and PMP-Based Culture Devices Over Existing Tissue Culture Devices.

Virtually all attempts to improve PS-based tissue culture devices for long-term BM culture have aimed to enhance cellular adhesion to tissue culture surfaces by modifying PS surfaces with ion plasma or by incorporating polypeptides, adhesion proteins or complex biomolecules. The latter approach includes coating of tissue culture surface with peptides (such as poly-D-lysine, RGD peptide) or adhesion proteins (such as collagen, laminin, fibronectin) or mucopolysaccharides (such as heparin sulfate, hyaluronidate and chondroitin sulfate). For example, the CORNING® CelIBIND surface (US. Pat. No. 6,617,152) aims to increase protein binding and cell adhesion by treating PS tissue culture surfaces with a higher energy microwave plasma in order to incorporate even more oxygen onto the PS surface to render it more hydrophilic and (electrically) negatively charged. Another approach is to create a 3-dimensional lattice or web in the culture substratum. All these approaches have the same objective of increasing protein binding and cell adhesion to the culture surface but share the common side effect of increasing macrophage (and monocyte and neutrophil) adhesion and activation. In contrast, our new culture devices aim to minimize macrophage adhesion and activation by employing a highly hydrophobic, non-charged culture surface with low protein- and low cell-binding capacities. As a result, the production of inflammatory mediators of macrophages and other phagocytes that are harmful to HSC and progenitors are greatly reduced and hematopoiesis can continue over a long period of time with expansion of HSC and robust de novo erythropoiesis and megakaryocytopoiesis, which are virtually impossible in traditional long-term bone marrow cultures using TC-PS culture devices.

### Advantage Over Ultra-Low-Binding Plates.

The goals of most efforts to “improve” tissue culture surfaces aim to achieve the exact opposite of what we strive for in this invention, i.e. low protein and low cell (phagocyte) binding. One rare exception is the “ultra-low attachment” tissue culture plate (“ULA” Surface; CORNING®). The ULA Surface employs a neutrally charged but hydrophilic “hydrogel” coating covalently linked to the PS base plate. The ULA Surface can also inhibit the attachment and activation of macrophages and neutrophils over the short term (36). However, direct comparison of the ULA plates with PE-coated plates in LTBMC (supplemented with mKL and mTPO and 10⁻⁶ M HC) showed that they performed much worse than PE-coated plates in supporting long-term hematopoiesis (3-4 weeks for ULA plates vs. 3-12 months for PE-coated plates). Serial observations showed that the ULA plates actually promoted the formation of large numbers of adherent, bizarre-shaped, multinucleated FBGC after 10-16 days of cultivation. It appears that macrophages in such cultures regarded the hydrogel as “foreign bodies” that must be ingested and destroyed through the formation of FBGC. It is also possible that the hydrogel eventually deteriorated and macrophages adhered to the exposed PS surface and formed FBGC as usual. In any case, the decline of hematopoiesis clearly accelerated with the appearance of adherent macrophages and FBGC in ULA culture plates.

Another low-protein-binding surface is the CORNING® NBS (Non-Binding Surface) with a nonionic, hydrophilic surface that minimizes nonspecific molecular interactions. NBS consists of a nonionic, hydrophilic ring structure coupled to the end of a polyethylene oxide-like linker, which is in turn linked to the PS surface. The salient property of NBS surface is very low protein and nucleic acid binding. They are designed for small fluid volume, high-throughput biochemical assays that require very low protein and low nucleic acid binding and are available only in 96-, 384- and 1536-well formats. Their compatibility with cell culture is not known and there are no NBS products for cell culture applications.

Another tissue culture device designed for low cell binding is the “PlusS” plate (Alpha Plus Scientific Corporation) that employs 2-methacryloyloxyethyl phosphocholine copolymer coating to inhibit cell binding. A direct comparison of PlusS plates with PE-coated plates showed that while the PlusS plates were very effective in inhibiting fibroblast and macrophage adhesion. Unlike the ULA plates, the PlusS plates did not induce the formation of FBGC. However, they still could not support long-term hematopoiesis. These findings indicate that inhibition of macrophage adhesion alone is not sufficient for creating an environment that is conducive to long-term hematopoiesis. The surface chemistry must also have a low potential for triggering macrophage activation.

### Advantage Over PolyHEMA-Coated Plates.

An economical approach for producing embryoid bodies (EB) from embryonic stem cells (ES) is by preventing ES cell adhesion to culture plates using PS or TC-PS dishes coated with poly(2-hydroxyethyl methacrylate), a.k.a. “polyHEMA” (37). PolyHEMA forms a hydrogel layer upon hydration. As discussed above, complete inhibition of cellular adhesion is not conducive to the survival of HSC and hematopoietic progenitors. Nor is it sufficient for preventing macrophage proinflammatory activation. Hydrogel can induce the formation of FBGC after a longer period of incubation. In addition, polyHEMA is not covalently linked to TC-PS and therefore will delaminate with time. Thus, polyHEMA-coated plates are not suitable for long-term BM cultures, either.

### Advantage Over Existing HSC Expansion Approaches Using Purified HSC as the Starting Populations.

Recently, it was reported that the replacement of serum albumin with polyvinyl alcohol (PVA) allowed amplification of purified mouse HSC (38). The method was premised on the hypothesis that serum albumin, including purified recombinant albumin, contained unidentified impurities and the replacement of albumin with chemically defined macromolecular substitutes such as PVA would solve the problem. Of relevance is that the cited work showed that mediators of innate immunity (essentially macrophage and neutrophil-produced inflammatory cytokines such as TNF_{α}, IL-1, IL-6 and MIP-1_{α}) were generated with the emergence of monocytes, macrophages and neutrophils in HSC cultures. Importantly, the conditioned media of such cultures inhibited HSC proliferation (38). While the cited work did not identify the inhibitors in the conditioned media, it alluded to the possibility that “mediators of native immunity” (proinflammatory cytokines and chemokines) were at work. This is in agreement with our findings. The method of HSC expansion in the cited work requires the use of highly purified (100% or nearly 100%) HSC as the starting population, apparently in order to avoid the effects of pro-inflammatory cytokines secreted by contaminating myeloid cells. In contrast, the LoMAC culture method described in this application works very well with unpurified hematopoietic cells. Judging from the beneficial effect of a larger starting population of unpurified BM cells (FIG. 12 vs. FIG. 13), it is possible that paracrine stimulation plays a positive role in LoMAC BM cultures. This is a great advantage since any protocol for in vitro HSC amplification for therapeutic applications is likely to involve the use of relatively large numbers of hematopoietic cells that are unpurified or at best semi-purified as the starting population. Thus, an HSC amplification method that works well with unpurified or partially purified human HSC preparations will save time, labor and materials and reduces HSC loss and microbial contamination.

### Advantage of a Slower Pace of HSC Expansion in BM LoMAC Cultures.

HSC expansion using LoMAC culture method in PE-coated or PMP-based culture devices takes place slowly. Our calculation indicates that HSC doubles in number about every 5-10 days in mouse BM LoMAC cultures (FIG. 13, solid line). This kinetics is similar to that of HSC in vivo. This slow expansion rate may actually be preferable as it provides more time for DNA repair in HSC and reduces mutations.

### Distinguishing Features of PE-Coated or PMP-Based Culture Devices.

The distinguishing feature of LoMAC culture method is the non-charged, hydrophobic, low protein-binding, low-cell-binding PE layer that covers the ENTIRE tissue culture surface that may come in contact with tissue culture medium and/or cells during incubation or manipulation of the cells. This includes the bottom AND sidewalls of tissue culture plates and the entire internal surface in the cases of tissue culture flasks and bags. (In contrast, most tissue culture devices with modified surfaces focus only on the bottom surface.) Alternatively, the entire tissue culture device can be fabricated from transparent, rigid polyolefins such as poly(4-methyl-1-pentene) (PMP) that share salient properties (no electrical charge, hydrophobicity, low protein- and cell-binding, no additional chemical bonds other than C—C and C—H that might contribute to macrophage activation) with the prototypical PE. PE and PMP are highly hydrophobic while most TC culture devices (TC-PS, other coatings, glass) have electrically charged, hydrophilic culture surfaces. As a result, PE and PMP bind different proteins and in smaller quantities than TC-PS. PE and PMP also have no special chemical moieties like the phenolic rings of PS that might participate in cell signaling via pattern-recognizing receptors. These differences translate into different adhesion and activation potentials for macrophages, the key orchestrator of inflammatory response and tissue repair.

The purposes of the polyolefin culture surface in LoMAC culture devices are two folds: (i) to reduce the adhesion of macrophages and other phagocytes to the culture surface and (ii) to prevent pro-inflammatory activation of macrophages and other phagocytes. Our data presented in Embodiments demonstrate that PE-coated or PMP-based devices are uniquely suited for long-term BM and HSC cultures due to their ability to foster a non-inflammatory or anti-inflammatory environment required for normal hematopoiesis in vitro. In addition to HSC expansion, these new devices can be used to re-examine the various activation states of macrophages, especially the “non-inflammatory’ state, in the absence of the ubiquitous but M1-biasing TC-PS tissue culture surface (17). They may also find applications in the cultivation or differentiation of other cell types such as T lymphocytes, NK cells, dendritic cells, embryoid bodies or organoids.

### CONCLUSION, RAMIFICATIONS AND SCOPE

The most important revelation arising from the work described in this application is that the TC-PS culture surface employed in virtually all current tissue culture devices is the root cause of HSC decline in traditional BM cultures and a PE-coated or PMP-based culture surface solves much of the problem. Another insight is that hematopoiesis must normally take place in a non-inflammatory or anti-inflammatory microenvironment. This has implications for all future strategies for ex vivo expansion of HSC or the production of red blood cells and platelets in vitro.

It has been widely assumed that a preformed stromal layer consisting of macrophages, endothelial cells, osteoblasts, osteoclasts and fibroblastoid stromal cells is critical for sustaining HSC and hematopoietic progenitors in culture, so are HC (10⁻⁵-10⁻⁶ M) and a hypothermic temperature (33° C.). It has also been assumed that increasing the cellular adhesion capacity of tissue culture devices will translate into better survival of HSC and hematopoietic progenitors. The BM LoMAC culture system described here using PE-coated or PMP-based culture devices begs to differ. An unavoidable consequence of using PE-coated or PMP-based culture devices is the complete elimination of fibroblastoid stromal cells and other anchorage-dependent stromal cells, the former are the main source of KL that HSC and their progenies need for survival (23-26). Therefore, it is necessary to supplement LoMAC cultures with KL. TPO is added since it independently and synergistically stimulates the survival and mitosis of HSC (32, 33). TPO has also been reported to reduce mutagensis in HSC by promoting non-homologous DNA repair (39). Although devoid of a stromal layer, the LoMAC system not only maintains but also amplifies HSC over a long period of time and de novo erythropoiesis and megakaryocytopoiesis occur robustly and continuously.

The sine qua non of the LoMAC culture system is the PE-coated or PMP-based culture device. Due to the low protein-binding capacity of PE and PMP and the absence of special chemical features (beside C—C and C—H bonds), few monocytes/macrophages adhere to the PE or PMP surface or become activated. It should be pointed out that the effects of PE and PMP on monocytes/macrophages apply to neutrophils as well.

Several lines of evidence suggest that the long-term culture-sustaining cells in BM LoMAC cultures are HSC: First, CAFC_{d35} has been shown to correlate with HSC numerically (28, 29); Second, our CAFC_{d35} data for day 0 BM samples (FIG. 12&13) are in line with the reported number of HSC in mouse BM (28-30, 34, 35); Third, BM LoMAC cultures produce all myeloid lineages spontaneously and can generate NK and B lymphoid precursors upon transfer to an appropriate environment; Fourth, all immature progenitors such as CLP, CFU-GEMM, CFU-E/Meg, BFU-E, CFU-GM and mature cells in BM LoMAC have limited lifespans (hours to days) and therefore must be regenerated continuously from a multipotent HSC; Fifth, the kinetics (4-5 weeks) of B and NK progenitor development in OP-9 co-cultures is consistent with their origin in HSC; Finally, HSC is the only known BM progenitor with the capacity to sustain multi-lineage lympho-hematopoiesis over 3-4 months. More recent studies using lethally irradiated CD45.1 vs 45.2 congenic mice confirmed the persistence and increased longevity of log-term repopulating HSC in LoMAC BM cultures while all such cells had disappeared after 20 days of cultivation using TC-PS culture devices. Thus, the in vivo bone marrow transplant data corroborate the in vitro findings.

One of the surprises in the current study is that most mouse HSC are eliminated within 2 weeks of the initiation of BM cultures in TC-PS-based culture devices even with the help of exogenous KL, TPO and HC (FIGS. 12-14). This should be cause for concern when one considers the fact that most current protocols for amplifying human HSC have a culture period of 10-14 days (40-43). Therefore, a switch to LoMAC culture devices will improve the outcomes significantly.

While the Embodiments described above focus on PE-covered or PMP-based culture devices, the same principle can be applied to other tissue culture devices such as individual culture dishes (ideally with the “super deep” design), multi-well cluster plates, culture flasks, culture tubes, culture bags and cell culture bioreactors. The critical element is a hydrophobic, low-protein-binding and low-macrophage-adhesion/activation culture surface. The material used to create the low macrophage-adhering/activation culture surface needs not to be limited to polyolefins and can be any material that has very low macrophage adhesion/activation potential and is nontoxic to HSC. However, polyolefins have the advantages of lower cost, durability and long safety records.

The application of PE-coated or PMP-based culture devices may not be limited to BM LoMAC cultures. They can be applied to situations where inhibition of cellular adhesion in general and of macrophage adhesion in particular is desirable. For example, the PE-coated or PMP-based culture devices can be used to cultivate ES cells or neuronal stem cells (NSC) or pancreatic β-islet cells or intestinal epithelia stem cells or tumor cells to encourage the formation of spheroid bodies or organoids by denying cellular adhesion to the culture surface. It is particularly useful where macrophages are present (e.g. in tissue explant) as contaminants or byproducts and their adhesion/activation has a negative effect on the cell types of interest. The PE-coated or PMP-based devices and the associated culture method can be applied to the cultivation and expansion of human cells such as cord blood stem/progenitor cells, bone marrow, peripheral blood stem/progenitor cells, T or B lymphocytes, NK cells and dendritic cells. In the case of T lymphocytes, NK and dendritic cells, the low cell adhesion property of PE-coated or PMP-based culture devices may facilitate the harvesting of cultured T lymphocytes, NK or dendritic cells without the use of proteolytic enzymes, calcium chelators or hypotonic solutions, all of which may damage or alter the properties of harvested cells or decrease yields.

### REFERENCES

1. Dexter, T. M., Allen, T. D., and Lajtha, L. G. 1977. Conditions controlling the proliferation of haematopoietic stem cells in vitro. J. Cell. Physiol. 91:335-344.
2. Greenberger, J. S. 1978. Sensitivity of corticosteroid-dependent insulin-resistant lipogenesis in marrow preadipocytes of obese diabetic (Db/db) mice. Nature 275:752-754.
3. Martinez, F. O., Sica, A., Mantovani, A., and Locati, M. 2008. Macrophage activation and polarization. Frontiers in Bioscience 13:453-461.
4. Wynn, T. A., and Vannella, K. M. 2016. Macrophage in tissue repair, regeneration, and fibrosis. Immunity 44:450-462.
5. Mills, C. D., and Ley, K. 2014 M1 and M2 macrophages: the chicken and the egg of immunity. J Innate Immun. 6:716-726.
6. Anderson, J. M., Rodriguez, A., and Chang, D. T. 2008. Foreign body reaction to biomaterials. Semin. Immunol. 20:86-100.
7. Broxmeyer, H. E., Williams, D. E., Lu, L., Cooper, S., Anderson, S. L., Beyer, G. S., Hoffman, R., and Rubin, B. Y. 1986. The suppressive influences of human tumor necrosis factors on bone marrow hematopoietic progenitor cells from normal donors and patients with leukemia: Synergism of tumor necrosis factor and interferon-γ. J. Immunol. 136:4487-4495.
8. Murase, T., Hotta, T., Saito, H., and Ohno, R. 1987. Effect of recombinant human tumor necrosis factor on the colony growth of human leukemia progenitor cells and normal hematopoietic progenitor cells. Blood 69:467-472.
9. Su, S-B., Mukaida, N., Wang, J-B., Zhang, Y., Takami, A., Nako, S., and Matsushima, K. 1997. Inhibition of immature erythroid progenitor cell proliferation by macrophage inflammatory protein-1α by interacting mainly with ha C—C chemokine receptor, CCR1. Blood 90:605-611.
10. Hino, M., Tojo, A., Myiazono, K., Urabe, A, and Takaku, F. 1988. Effects of type β transforming growth factors on haematopoietic progenitor cells. Br. J. Haematol. 70:143-147.
11. Spooncer, E., Lord, B. I., and Dexter, T. M. 1985. Defective ability to self-renew in vitro of highly purified primitive haematopoietic cells. Nature 316:62-64.
12. Petursson, S. R., and Chervenick, P. A. 1985. Megakaryocytopoiesis and granulopoiesis of W/Wv mice studied in long-term bone marrow cultures. Blood 65:1460-1468.
13. Yang, Y. C., Tsai, S., Wong, G. G., and Clark, S. C. 1988. Interleukin-1 regulation of hematopoietic growth factor production by human stromal fibroblasts. J. Cell. Physiol. 134:292-296.
14. Sieff, C. A., Tsai, S., and Faller, D. V. 1987. Interleukin-1 induces cultured human endothelial cell production of granulocyte-macrophage colony stimulating factor. 1987. J. Clin. Invest. 79:48-51.
15. Henson, P. M. 1971. The immunologic release of constituents from neutrophil leukocytes. II. Mechanisms of release during phagocytosis, and adherence to nonphagocytosable surfaces. J. Immunol. 107:1547-1557.
16. Lee, M. H., Ducheyne, P., Lynch, L., Boettiger, D., and Composto, R. J. 2006. Effect of biomaterial surface properties on fibronectin-alpha5beta1 integrin interaction and cellular attachment. Biomaterials 27:1907-1916.
17. Rostam, H. M., Singh, S., Salazar, F., Magennis, P., Hook, A., Singh, T., Vrana, N. E., Alexander, M. R., and Ghaemmaghami, A. M. 2016. The impact of surface chemistry modification on macrophage polarization. Immunobiol. 22:1237-1246.
18. von Pechmann, H. 1898. Ueber Diazomethan and Nitrosoacylamine. Berichte der Deutschen Chemischen Gesellschaft zu Berlin 31:2640-2646.
19. Simon, E. 1839. Ueber den fluessigen Storax (Styrax liquidus). Annalen der Chemie 31:265-277.
20. Nakano, T., Kodama, H. and Honjo, T. Generation of lymphohematopoietic cells from embryonic stem cells in culture. 1994. Science 265:1098-1101.
21. Ralph, P. et al. 1976. Lysozyme synthesis by established human and murine histiocytic lymphoma cell lines. J. Exp. Med. 143:1528-1533.
22. Ralph, P., Nakoinz, I. 1977. Antibody-dependent killing of erythrocyte and tumor targets by macrophage-related cell lines: enhancement by PPD and LPS. J. Immunol. 119:950-954.
23. Williams, D. E., Eisenman, J., Baird, A., Rauch, C. and Lyman, S.D. 1990. Identification of a ligand for the c-kit proto-oncogene. Cell 63:167-174.
24. Martin, F. H., Suggs, S. V., Langley, K. E., Lu, H. S., and Zsebo, K. M. 1990. Primary structure and functional expression of rat and human stem cell factor DNAs. Cell 63:203-211.
25. Huang, E., Nocka, K., Beier, D. R., Chu, T-Y., Besmer, P. 1990. The hematopoietic growth factor KL is encoded by the SI locus and is the ligand of the c-kit receptor, the gene product of the W locus. Cell 63:225-233.
26. Flanagan, J. G., and Leder, P. 1990. The kit ligand: A cell surface molecule altered in steel mutant fibroblasts. Cell 63:185-194.
27. Lok, S., Kaushansky, K., . . . and Foster, D. 1994. Cloning and expression of murine thrombopoietin cDNA and stimulation of platelet production in vivo. Nature 369:565-568.
28. Ploemacher, R., van der Sluijs, J., Voerman, J. S. A., and Bron, N. H. C. 1989. An in vitro limiting-dilution assay of long-term repopulating hematopoietic stem cell in the mouse. Blood. 74: 2755-2763.
29. Ploemacher, R. E., van der Sluijs, J. P., van Beurden, C. A. J., Baert, M. R. M., and Chan, P. L. 1991. Use of limiting-dilution type long-term marrow culture in frequency analysis of marrow-repopulating and spleen colony-forming hematopoietic stem cells in the mouse. Blood 78:2527-2533.
30. Sieburg, H. B., Cho, R. H., and Mueller-Sieburg, C. E. 2002. Limiting dilution analysis for estimating the frequency of hematopoietic stem cells: Uncertainty and significance. Exp. Hematology 30:1436-1443.
31. DeHart, S. L., Heikens, M. J., and Tsai, S. 2005. Jagged2 promotes the development of natural killer cells and the establishment of functional natural killer cell lines. Blood 105:3521-3527.
32. Ku, H., Yonemura, Y., and Kaushansky, K., et al. 1996. Thrombopoietin, the ligand for the Mpl receptor, synergizes with steel factor and other early acting cytokines in supporting proliferation of primitive hematopoietic progenitors of mice. Blood 87:4544-4551.
33. Sitnicka, E., Lin, N., Priestley, G. V., et al. 1996. The effect of thrombopoietin on the proliferation and differentiation of murine hematopoietic stem cells. Blood 87:4998-5005.
34. Spangrude, G. J., Heimfield, S., and Weissman, I. L. 1988. Purification and characterization of mouse hematopoietic stem cells. Science 241:58-62.
35. Szilvassy, S. J., Humphries, R. K., Lansdorp, P. M., Eaves, C. A., and Eaves, C. J. 1990. Quantitative assay for totipotent reconstituting hematopoietic stem cells by a competitive repopulation strategy. Proc. Natl. Acad. Scie. USA 87:8736-8740.
36. Shen, M, and Horbett, T. A. 2001. The effects of surface chemistry and adsorbed proteins on monocyte/macrophage adhesion to chemically modified polystyrene surfaces. J. Biomed. Mat. Res. 57:336-345.
37. Wichterle, O., and Lim, D. 1960. Hydrophilic gels for biological use. Nature 185: 117-118.
38. Wilkinson, A. C., Ishida, R., Kikuchi, M., Sudo, K., Morita, M., Crisostomo, R. V., Yamamoto, R., Loh, K. M., Nakamura, Y., Watanabe, M., Nakauchi, H., and Yamazaki, S. 2019. Long-term ex vivo haematopoietic-stem-cell expansion allows nonconditioned transplantation. Nature 571: 117-121.
39. de Laval, B., Pawlikowska, P., Petit-Cocault, L., Bilhou-Nabera, C., Aubin-Houzelstein, G., Souyri, M., Pouzoulet, F., Gaudry, M., and Proteu, F. 2013. Thrombopoietin-increased DNA-PK-dependent DNA repair limits hematopoietic stem and progenitor cell mutagenesis in response to DNA damage. Cell Stem Cell 12:37-48.
40. Rogers, I. M., Yamanaka, N., and Casper, R. F. 2008. A simplified procedure for hematopoietic stem cell amplification using a serum-free culture system. Biol. Of Blood and Marrow Transplantation 14:927-937.
41. de Lima, M., McNiece, I., Robinson, S. N., Munsell, M., Eapen, M., Horowitz, M., Alousi, A., Saliba, R., McMannis, J. D., Kaur, I., et al. 2012. Cord-blood engraftment with ex vivo mesenchymal-cell coculture. N. Engl. J. Med. 367:2305-2315.
42. Horwitz, M. E., Chao, N. J., Rizzieri, D. A., Long, G. D., Sullivan, K. M., Gasparetto, C, Chute, J. P., Morris, A., McDonald, C., Waters-Pick, B., et al. 2014. Umbilical cord blood expansion with nicotinamide provides long-term multilineage engraftment. J. Clin. Invest. 124:3121-3128.
43. Wagner, J. E., Jr., Brunstein, C. G., Boitano, A. E., DeFor, T. E., McKenna, D., Sumstad, D., Blazer, B. R., Toler, J., Le, C., and Jones, J. et al. 2016. Phase I/II trial of StemRegenin-1 expanded umbilical cord blood hematopoietic stem cells supports testing as a stand-alone graft. Cell Stem Cell 18:144-155.

What is claimed is:

1. A cell culture device fabricated from materials with a twenty-or-more-fold lower capacity to stimulate macrophage adhesion and their pro-inflammatory activation, including the formation of foreign body giant cells, than polystyrene or tissue culture-treated polystyrene.

2. The device in claim 1 that is fabricated from low-density polyethylene with a density range of 0.910-0.940 g/cm³.

3. A cell culture device in which all internal surfaces in temporary or permanent contact with the cultured cells are covered with a bonded layer of materials with a twenty-or-more-fold lower capacity to stimulate macrophage adhesion and their proinflammatory activation, including the formation of foreign body giant cells, than polystyrene or tissue culture-treated polystyrene.

4. The device in claim 3 in which the internal surfaces are covered with a layer of low-density polyethylene with a density range of 0.910-0.940 g/cm³.

5. A removable cell culture insert in which all internal surfaces in temporary or permanent contact with the cultured cells are composed of or covered with a layer of materials with a twenty-or-more-fold lower capacity to stimulate macrophage adhesion and their proinflammatory activation, including the formation of foreign body giant cells, than polystyrene or tissue culture-treated polystyrene.

6. The insert in claim 5 in which all internal surfaces in temporary or permanent contact with the cultured cells are composed of or covered with a layer of low-density polyethylene with a density range of 0.910-0.940 g/cm³.
